(19) United States
(12) Patent Application Publication
(10) Pub. No.: US 2018/0170903 A1
(43) Pub. Date: Jun. 21, 2018

(54) PYRIDIN-3-YL ACETIC ACID DERIVATIVES AS INHIBITORS OF HUMAN IMMUNODEFICIENCY VIRUS REPLICATION

(71) Applicant: ViiV HEALTHCARE UK (NO.5) LIMITED, Brentford Middlesex (GB)

(72) Inventors: John F. KADOW, Wallingford, CT (US); B. Narasimhulu NAIDU, Wallingford, CT (US); Tao WANG, Wallingford, CT (US); Zhiwei YIN, Wallingford, CT (US)

(21) Appl. No.: 15/578,906

(22) PCT Filed: Jul. 6, 2016
(86) PCT No.: PCT/IB2016/054049

§ 371 Date: Dec. 1, 2017

Related U.S. Application Data
(60) Provisional application No. 62/188,852, filed on Jul. 6, 2015.

(51) Int. Cl. C07D 401/04; A61P 31/18; A61K 31/5365; A61K 31/4545; A61K 31/5377; C07D 405/14; C07D 401/14; C07D 409/14; A61K 31/4725

(52) U.S. Cl. CPC C07D 401/04; A61P 31/18; A61K 31/5365; A61K 31/4545; A61K 31/4725; C07D 405/14; C07D 401/14; C07D 409/14; A61K 31/5377

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS.

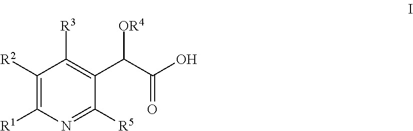

### CROSS REFERENCE TO RELATED INVENTION

This application claims the benefit of U.S. provisional application Ser. No. 62/188,852 filed Jul. 6, 2015.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, and WO2015126726.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

By virtue of the present invention, it is now possible to provide compounds that are novel and are useful in the treatment of HIV. Additionally, the compounds may provide advantages for pharmaceutical uses, for example, with regard to one or more of their mechanism of action, binding, inhibition efficacy, target selectivity, solubility, safety profiles, or bioavailability.

The invention also provides pharmaceutical compositions comprising the compounds of the invention, including pharmaceutically acceptable salts thereof, and a pharmaceutically acceptable carrier, excipient, and/or diluent.

In addition, the invention provides methods of treating HIV infection comprising administering a therapeutically effective amount of the compounds of the invention to a patient.

In addition, the invention provides methods for inhibiting HIV integrase.

Also provided in accordance with the invention are methods for making the compounds of the invention.

The present invention is directed to these, as well as other important ends, hereinafter described.

### DESCRIPTION OF THE INVENTION

Unless specified otherwise, these terms have the following meanings.

“Alkyl” means a straight or branched saturated hydrocarbon comprised of 1 to 10 carbons, and preferably 1 to 6 carbons.

“Alkenyl” means a straight or branched alkyl group comprised of 2 to 10 carbons with at least one double bond and optionally substituted with 0-3 halo or alkoxy group.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic groups include, but are not limited to indanyl, indenyl, naphthyl, phenyl, tetrahydronaphthyl and cyclopropylphenyl. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Cycloalkyl” means a monocyclic ring system composed of 3 to 7 carbons.

“Halo” includes fluoro, chloro, bromo, and iodo.

“Haloalkyl” and “haloalkoxy” include all halogenated isomers from monohalo to perhalo.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, bezodioxolyl, benzoisothiazole, benzothiazole, benzothiadiazole, benzothiophene, benzoxazole, carbazole, chroman, dihalobezodioxolyl, dihydrobenzofuran, dihydrobenzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n=1-5 and that R^{Z} is chosen from benzimidazole, imidazole, indazole, isooxazole, phenyl-pyrazole, pyridine, quinoline, thiazole, triazole, triazolone, oxadiazole.

Terms with a hydrocarbon moiety (e.g. alkoxy) include straight and branched isomers for the hydrocarbon portion with the indicated number of carbon atoms.

Bonding and positional bonding relationships are those that are stable as understood by practitioners of organic chemistry.

Parenthetic and multiparenthetic terms are intended to clarify bonding relationships to those skilled in the art. For example, a term such as ((R)alkyl) means an alkyl substituent further substituted with the substituent R.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In an aspect of the invention, there is provided a compound of Formula I:

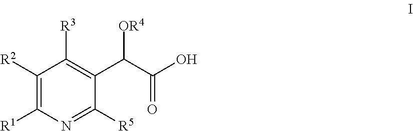

wherein:
R¹ is selected from hydrogen or alkyl;
R² is selected from ((R⁶O)CR⁹R¹⁰)phenyl, ((R⁶S)CR⁹R¹⁰)phenyl, or (((R⁶)(R⁷)N)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is selected from alkyl, cycloalkyl, (cycloalkyl)alkyl, (R⁸)C₁₋₃-alkyl, or (Ar¹)C₀₋₃-alkyl;
R⁷ is selected from hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; or N(R⁶)(R⁷) taken together is tetrahydroisoquinolinyl;
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

For a particular compound of Formula I, the scope of any instance of a variable substituent, including R¹, R², R³, R⁴, R⁵, R⁶, R⁷, R⁸, R⁹, R¹⁰, Ar¹ and Ar² can be used independently with the scope of any other instance of a variable substituent. As such, the invention includes combinations of the different aspects.

In an aspect of the invention, R¹ is alkyl; R² is (((R⁶)(R⁷)N)CR⁹R¹⁰)phenyl; R³ is piperidinyl substituted with 0-3 substituents selected from cyano, halo, alkyl, haloalkyl, alkoxy, or haloalkoxy; R⁹ is hydrogen; R¹⁰ is hydrogen; and Ar¹ is phenyl substituted with 0-3 substituents selected from halo, alkyl, haloalkyl, alkoxy, haloalkoxy, carboxy, and alkoxycarbonyl.

In an aspect of the invention, R⁶ is (Ar¹)C₁₋₃-alkyl; and R⁸ is amino, alkylamino, or dialkylamino.

In an aspect of the invention, R² is ((R⁶O)CR⁹R¹⁰)phenyl or ((R⁶S)CR⁹R¹⁰)phenyl.

In an aspect of the invention, R² is (((R⁶)(R⁷)N)CR⁹R¹⁰)phenyl.

In an aspect of the invention, R⁶ is (Ar¹)C₀₋₃-alkyl;

R⁷ is hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; and
R⁹ and R¹⁰ are hydrogen.

In an aspect of the invention, R⁹ and R¹⁰ are hydrogen.

In an aspect of the invention, there is provided a compound of Formula I:

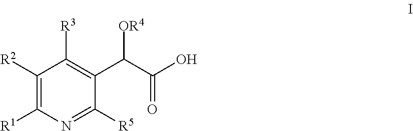

wherein:
R¹ is selected from hydrogen or alkyl;
R² is selected from ((R⁶O)CR⁹R¹⁰)phenyl or ((R⁶S)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is selected from alkyl, cycloalkyl, (cycloalkyl)alkyl, (R⁸)C₁₋₃-alkyl, or (Ar¹)C₀₋₃-alkyl;
R⁷ is selected from hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; or N(R⁶)(R⁷) taken together is tetrahydroisoquinolinyl;
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

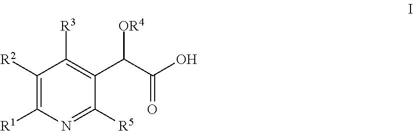

wherein:
R¹ is selected from hydrogen or alkyl;
R² is (((R⁶)(R⁷)N)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is (Ar¹)C₀₋₃-alkyl;
R⁷ is hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; and
R⁹ and R¹⁰ are hydrogen.
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

Preferred compounds in accordance with the present invention include the following:
(S)-2-(tert-Butoxy)-2-(4-(4,4-dimethylpiperidin-1-yl)-5-(4-(((4-fluoro-3-methylbenzyl)amino)methyl)phenyl)-2,6-dimethylpyridin-3-yl)acetic acid;
(S)-2-(tert-Butoxy)-2-(4-(4,4-dimethylpiperidin-1-yl)-2,6-dimethyl-5-(4-(morpholinomethyl)phenyl)pyridin-3-yl)acetic acid;
(2 S)-2-(tert-Butoxy)-2-(4-(4,4-dimethylpiperidin-1-yl)-5-(4-(((4-fluorobenzyl)((tetrahydrofuran-2-yl)methyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((4-fluorobenzyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((4-fluorophenethyl)(methyl)amino)methyl)phenyl)-2,6-dimethylpyridin-3-yl)acetic acid;
(S)-2-(tert-Butoxy)-2-(5-(4-(((3,3-dimethylbutyl)amino)methyl)phenyl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-5-(4-(((4-fluorobenzyl)(methyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((4-methoxyphenethyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((2-methoxyphenethyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((4-fluorobenzyl)(methoxy)amino)methyl)phenyl)-2,6-dimethylpyridin-3-yl)acetic acid;
(S)-2-(tert-Butoxy)-2-(4-(4,4-dimethylpiperidin-1-yl)-5-(4-(((4-fluorophenethyl)amino)methyl)phenyl)-2,6-dimethylpyridin-3-yl)acetic acid;
(S)-2-(tert-Butoxy)-2-(5-(4-(((3,4-dichlorobenzyl)amino)methyl)phenyl)-4-(4,4-dimethylpiperidin-1l-yl)-2,6-dimethylpyridin-3-yl)acetic acid;
(S)-2-(tert-Butoxy)-2-(5-(4-(((2-cyclohexylethyl)amino)methyl)phenyl)-4-(4,4-dimethylpiperidin-1-yl)-2,6-dimethylpyridin-3-yl)acetic acid;
(S)-2-(5-(4-((Benzylamino)methyl)phenyl)-4-(4,4-dimethylpiperidin-l-yl)-2,6-dimethylpyridin-3-yl)-2-(tert-butoxy)acetic acid;
(S)-2-(tert-Butoxy)-2-(5-(4-(((4-chlorobenzyl)amino)methyl)phenyl)-4-(4,4-dimethylpiperidin-1l-yl)-2,6-dimethylpyridin-3-yl)acetic acid;
(S)-2-(tert-Butoxy)-2-(4-(4,4-dimethylpiperidin-1l-yl)-2,6-dimethyl-5-(4-(((4-methylbenzyl)amino)methyl)phenyl)pyridin-3-yl)acetic acid;
(S)-2-(tert-Butoxy)-2-(5-(4-(((cyclohexylmethyl)amino)methyl)phenyl)-4-(4,4-dimethylpiperidin-1l-yl)-2,6-dimethylpyridin-3-yl)acetic acid;
(S)-2-(tert-Butoxy)-2-(4-(4,4-dimethylpiperidin-1-yl)-5-(4-(((4-(methoxycarbonyl)benzyl)amino)methyl)phenyl)-2,6-dimethylpyridin-3-yl)acetic acid;
(S)-4-(((4-(5-(tert-Butoxy(carboxy)methyl)-4-(4,4-dimethylpiperidin-1-yl)-2,6-dimethylpyridin-3-yl)benzyl)amino)methyl)benzoic acid;
(S)-2-(tert-Butoxy)-2-(4-(4,4-dimethylpiperidin-1-yl)-5-(4-((N-(4-fluorobenzyl)cyclopentanecarboxamido)methyl)phenyl)-2,6-dimethylpyridin-3-yl)acetic acid;
(S)-2-(tert-Butoxy)-2-(4-(4,4-dimethylpiperidin-1l-yl)-5-(4-((N-(4-fluorobenzyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)propionamido)methyl)phenyl)-2,6-dimethylpyridin-3-yl)acetic acid
(S)-2-(tert-Butoxy)-2-(4-(4,4-dimethylpiperidin-1-yl)-5-(4-((N-(4-fluorobenzyl)isobutyramido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)acetamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)pivalamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2-methoxybenzamido)methyl)phenyl)-2,6-dimethylpyridin-3-yl)acetic acid;
(S)-2-(tert-Butoxy)-2-(4-(4,4-dimethylpiperidin-1-yl)-5-(4-((2-fluoro-N-(4-fluorobenzyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((4-fluoro-N-(4-fluorobenzyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2,5-dimethylfuran-3-carboxamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2-phenoxyacetamido)methyl)phenyl)-2,6-dimethylpyridin-3-yl)acetic acid;
(S)-2-(5-(4-((((Benzyloxy)carbonyl)(4-fluorobenzyl)amino)methyl)phenyl)-4-(4,4-dimethylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-5-(4-(((4-fluorobenzyl)(methoxycarbonyl)amino)methyl)phenyl)-2,6-dimethylpyridin-3-yl)acetic acid;
(S)-2-(tert-Butoxy)-2-(4-(4,4-dimethylpiperidin-1-yl)-5-(4-(((ethoxycarbonyl)(4-fluorobenzyl)amino)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)methyl sulfonamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)pyrrolidine-1-carboxamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)phenyl sulfonamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-3-methoxybenzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-3-(trifluoromethyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2-(trifluoromethyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)cyclopropanecarboxamido)methyl)phenyl)-2,6-dimethylpyridin-3-yl)acetic acid;
(S)-2-(tert-Butoxy)-2-(4-(4,4-dimethylpiperidin-1-yl)-5-(4-((3-fluoro-N-(4-fluorobenzyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)cyclobutanecarboxamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2-methylbenzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)thiophene-2-carboxamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-4-methoxybenzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-4-(trifluoromethyl)benzamido)methyl)phenyl)-2,6-dimethylpyridin-3-yl)acetic acid;
(S)-2-(tert-Butoxy)-2-(4-(4,4-dimethylpiperidin-1-yl)-5-(4-((N-(4-fluorobenzyl)-2,4,6-trimethylphenylsulfonamido)methyl)phenyl)-2,6-dimethylpyridin-3-yl)acetic acid;
(S)-2-(tert-Butoxy)-2-(4-(4,4-dimethylpiperidin-1-yl)-5-(4-(hydroxymethyl)phenyl)-2,6-dimethylpyridin-3-yl)acetic acid;
(S)-2-(tert-Butoxy)-2-(4-(4,4-dimethylpiperidin-1-yl)-2,6-dimethyl-5-(4-((3-(trifluoromethyl)phenoxy)methyl)phenyl)pyridin-3-yl)acetic acid;
(S)-2-(tert-Butoxy)-2-(5-(4-(tert-butoxymethyl)phenyl)-4-(4,4-dimethylpiperidin-1-yl)-2,6-dimethylpyridin-3-yl)acetic acid;
(S)-2-(tert-Butoxy)-2-(5-(4-((4-chloro-3-methylphenoxy)methyl)phenyl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-5-(4-(((4-fluorobenzyl)oxy)methyl)phenyl)-2,6-dimethylpyridin-3-yl)acetic acid;
(S)-2-(tert-Butoxy)-2-(4-(4,4-dimethylpiperidin-1-yl)-5-(4-(((4-fluorobenzyl)thio)methyl)phenyl)-2,6-dimethylpyridin-3-yl)acetic acid; and
(S)-2-(tert-Butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetic acid; and
pharmaceutically acceptable salts thereof.

The compounds of the invention herein described may typically be administered as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

The compounds of this invention desireably have activity against HIV. Accordingly, another aspect of the invention is a method for treating HIV infection in a human patient comprising administering a therapeutically effective amount of a compound of Formula I, or a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, HIV capsid inhibitors, anti-infectives, and immunomodulators, such as, for example, PD-1 inhibitors, PD-L1 inhinitors, antibodies, and the like. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

Examples of nucleoside HIV reverse transcriptase inhibitors include abacavir, didanosine, emtricitabine, lamivudine, stavudine, tenofovir, zalcitabine, and zidovudine.

Examples of non-nucleoside HIV reverse transcriptase inhibitors include delavirdine, efavirenz, etrivirine, nevirapine, and rilpivirine.

Examples of HIV protease inhibitors include amprenavir, atazanavir, darunavir, fosamprenavir, indinavir, lopinavir, nelfinavir, ritonavir, saquinavir and, tipranavir.

An example of an HIV fusion inhibitor is enfuvirtide or T-1249.

An example of an HIV entry inhibitor is maraviroc.

Examples of HIV integrase inhibitors include dolutegravir, elvitegravir, or raltegravir.

An example of an HIV attachment inhibitor is fostemsavir.

An example of an HIV maturation inhibitor is BMS-955176, having the following structure:

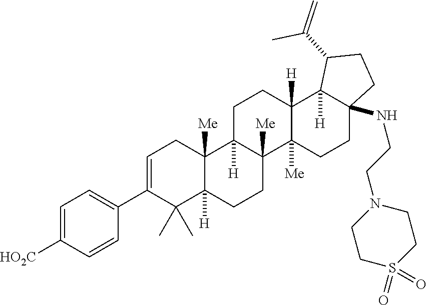

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside |
| | | reverse transcriptase |
| | | inhibitor) |
| COMPLERA ® | Gilead | HIV infection, AIDS, |
| | | ARC; combination |
| | | with emtricitabine, rilpivirine, |
| | | and tenofovir disoproxil |
| | | fumarate |
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® | Gilead | HIV infection, |
| (Emtricitabine) (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| SELZENTRY ™ | Pfizer | HIV infection |
| Maraviroc; (UK 427857) | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |
| TIVICAY ® | | |
| dolutegravir | | |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potassium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized from an appropriately substituted heterocycle I-1 according to Scheme I. Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate I-4. Coupling of amines 1-6 with intermediate 1-5 in the presence of an organic base such as Hunig's base provided intermediate I-7. Chiral Lewis acid such as I-8 mediated reduction of ketoester I-7 with catecholborane furnished the chiral alcohol I-9. Tertiary butylation of alcohol I-9 by well-known conditions, including but not limited to isobutylene and perchloric acid, gave intermediate I-10. Intermediate I-10 was conveniently transformed to intermediate I-11 using conditions well-known in the art, including but not limited to the Suzuki coupling between intermediate I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished carboxylic acid I-12.

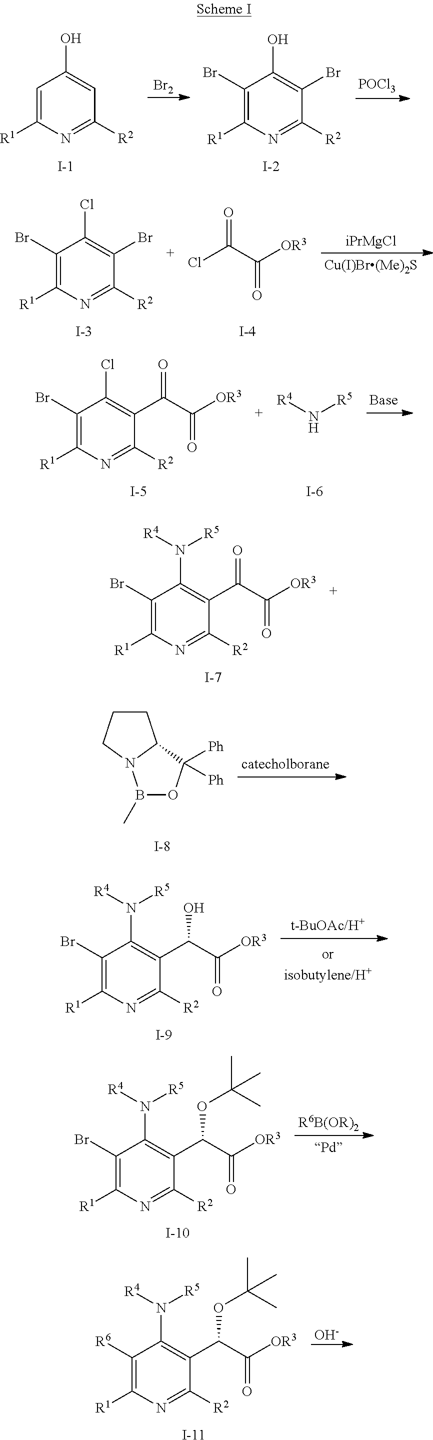

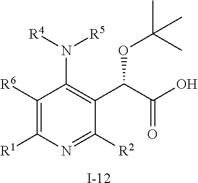

Intermediate I-10 conveniently transformed to intermediate II-2 using conditions well-known in the art, including but not limited to the Suzuki coupling between intermediate I-10 and boronic acid derivative II-1. The boronic acid derivatives II-1 are well-known in the art and are commercially available or are prepared by reactions well-known to those skilled in the art. The aldehyde II-2 and the amine II_3 were coupled using reductive alkylation conditions well know to those skilled in the art, including but not limited to NaCNBH₄/ZnCl₂ provided intermediate II-4. Hydrolysis of intermediate II-4 by using conditions well-known in the literature furnished carboxylic acid II-5.

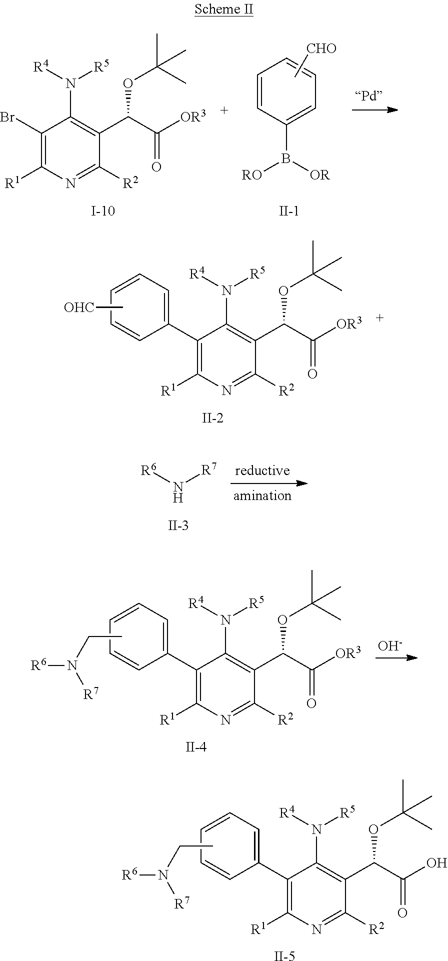

The compounds described herein were purified by the methods well known to those skilled in art by normal phase column chromatography on silica gel column using appropriate solvent system described. Preparative HPLC purifications mentioned in this experimentation section were carried out gradient elution either on Sunfire Prep C18 ODB column (5 μm; 19 or 30×100 mm) or Waters Xbridge column (5 μM; 19 or 30×100 mm) using the following mobile phases: Mobile phase A: 9:1 H₂O/acetonitrile with 10 mM NH₄OAc and mobile phase B: A: 9:1 acetonitrile/H₂O with: 10 mM NH₄OAc; or mobile phase A: 9:1 H₂O/acetonitrile with 0.1% TFA and mobile phase B: A: 9:1 acetonitrile/H₂O with: 0.1% TFA; or mobile phase A: water with 20 mM NH₄OAc and mobile phase B: 95:5 MeOH/H₂O with 20 mM NH₄OAc.

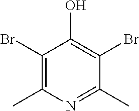

### 3,5-Dibromo-2,6-dimethylpyridin-4-ol

A 3-neck R.B-flask equipped with mechanical stirrer, addition funnel and condenser is charged with 2,6-dimethylpyridin-4-ol (100 g, 812 mmol), CH₂Cl₂ (1000 mL) and MeOH (120 mL). To the resulting light brown or tan solution was added tert-BuNH₂ (176 ml, 1665 mmol), cooled in water bath maintained between 5-10° C. (ice-water) and added drop wise Br₂ (84 ml, 1624 mmol) over 70 min. After the addition was complete cold bath was removed and stirred for 1.5 h at rt. Then, the light orange slurry was filtered and the filter cake was washed with ether (250 mL) and dried to afford 3,5-dibromo-2,6-dimethylpyridin-4-ol, hydrobromide (280.75 g, 776 mmol, 96% yield) as white solid which was used in the next step without further purification. ¹H NMR (500 MHz, DMSO-d₆) δ 12.08 (br. s., 1H), 2.41 (s, 6H). LCMS (M+H)=281.9.

### Alternative Procedure:

Bromine (72.8 mL, 1.4 mol) was added via addition funnel over 60 min to a mechanically stirred cold (ice-water bath) solution of 2,6-dimethylpyridin-4-ol (87 g, 706 mmol) and 4-methylmorpholine (156 mL, 1.4 mol) in dichloromethane (1 L) and methanol (100 mL) and then stirred for 2 h at rt. Additional bromine (˜15 mL) was added based on monitoring by LCMS. The product was filtered, washed with ether, and dried under vacuum to give 3,5-dibromo-2,6-dimethylpyridin-4-ol 176.8 g (88%).

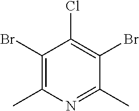

### 3,5-Dibromo-4-chloro-2,6-dimethyl-pyridine

Triethylamine (28.8 mL, 206 mmol) was added to a nitrogen purged solution of 3,5-dibromo-2,6-dimethylpyridin-4-ol (58 g, 206 mmol) and phosphorous oxychloride (57.7 mL, 619 mmol) in chloroform (450 mL) and stirred for 1 h at rt, then 3 h at 80° C. The reaction was removed from heating and immediately concentrated under house vacuum; then under high vacuum. The appearance was a cream colored solid, which was azeotroped with toluene (2×100 mL); treated with ice (200 g) for 10 min and carefully neutralized with NaHCO₃ (powder), and 1N NaOH solution, and extracted with DCM (2×400 mL). The combined organic layers were dried (MgSO₄), concentrated, and a beige solid was obtained that was washed with hexanes and dried under high vacuum to give 3,5-dibromo-4-chloro-2,6-dimethyl-pyridine 52.74 g (85.1%). Concentration of the hexanes gave 3.5 g of less pure product. ¹H NMR (500 MHz, CDCl₃) δ 2.59 (s, 6H). LCMS (M+H)=300.0.

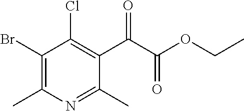 [SMILES: CCOC(=O)C(=O)c1c(C)nc(C)c(Br)c1Cl]

### Ethyl 2-(5-bromo-4-chloro-2,6-dimethylpyridin-3-yl)-2-oxoacetate

To a stirred mixture of 3,5-dibromo-4-chloro-2,6-dimethylpyridine (14.94 g, 49.9 mmol) and Cu(I)Br Me2S (0.513 g, 2.495 mmol) in THF (50 mL) was added drop wise 2M iPrMgCl/THF (26.2 ml, 52.4 mmol) at −30° C. over 5 min. Then, the resulting slurry was warmed to −10° C. over 30 min and stirred for 30 min. The homogeneous brown reaction mixture was rapidly transferred via cannula to a solution of ethyl 2-chloro-2-oxoacetate (6.14 ml, 54.9 mmol, degassed for 5 min by bubbling N2 through the solution) in THF (50 mL) maintained at −30° C. The resulting reaction mixture was stirred (1.5 h) while warming to 0° C. Then, taken up in to Et₂O (200 mL), washed with 1:1 sat Na₂CO₃/1M NH₄Cl (3×50 mL), dried (MgSO₄), filtered and concentrated to give brown viscous oil. Flash chromatography using 2.5, 5 and 7.5% EtOAc/Hex afforded ethyl 2-(5-bromo-4-chloro-2,6-dimethylpyridin-3-yl)-2-oxoacetate (14.37 g, 44.8 mmol, 90% yield) as white solid. ¹H NMR (400 MHz, CDCl₃) δ 4.42 (q, J=7.0 Hz, 2H), 2.76 (s, 3H), 2.46 (s, 3H), 1.41 (t, J=7.2 Hz, 3H). LCMS (M+H)=322.1.

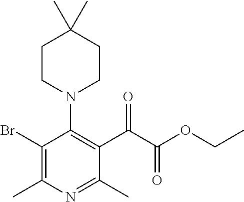 [SMILES: CCOC(=O)C(=O)c1c(C)nc(C)c(Br)c1N1CCC(C)(C)CC1]

### Ethyl 2-(5-bromo-4-(4,4-dimethylpiperidin-1-yl)-2,6-dimethylpyridin-3-yl)-2-oxoacetate

To a solution of 4,4-dimethylpiperidine (1.245 g, 11.00 mmol) and DIEA (3.49 ml, 20.00 mmol) in anhydrous CH₃CN (40 mL) was added ethyl 2-(5-bromo-4-chloro-2,6-dimethylpyridin-3-yl)-2-oxoacetate (3.21 g, 10 mmol) at rt. The resulting mixture was placed in a pre-heated oil bath (80° C.). After 22 h, the reaction mixture was concentrated and the residue was purified by flash chromatography using 1-lit each 2.5, 5, 7.5 and 10% EtOAc/Hex to afford ethyl 2-(5-bromo-4-(4,4-dimethylpiperidin-1-yl)-2,6-dimethylpyridin-3-yl)-2-oxoacetate (2.846 g, 7.16 mmol, 71.6% yield) as yellow solid. ¹H NMR (500 MHz, CDCl₃) δ 4.37 (q, J=7.1 Hz, 2H), 3.67-2.75 (br.s., 4H), 2.71 (s, 3H), 2.44 (s, 3H), 1.42 (t, J=7.1 Hz, 3H), 1.38 (t, J=5.6 Hz, 4H), 1.00 (s, 6H). LCMS (M+H)=399.4.

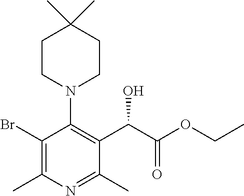 [SMILES: CCOC(=O)[C@@H](O)c1c(C)nc(C)c(Br)c1N1CCC(C)(C)CC1]

### (S)-Ethyl 2-(5-bromo-4-chloro-2,6-dimethylpyridin-3-yl)-2-hydroxyacetate

To stirred yellow solution of ethyl 2-(5-bromo-4-(4,4-dimethylpiperidin-1-yl)-2,6-dimethylpyridin-3-yl)-2-oxoacetate (2.25 g, 5.66 mmol) and (R)-1-methyl-3,3-diphenylhexahydropyrrolo[1,2-c][1,3,2]oxazaborole (0.314 g, 1.133 mmol) in toluene (30 mL) at −35° C. was added drop wise 50% catecholborane (1.819 ml, 8.49 mmol) over 10 min. The reaction mixture was slowly warmed to −15° C. over 1 h and then left for 2 h at −15° C. Then, diluted with EtOAc (100 mL), washed with sat Na₂CO₃ (4×25 mL) by vigorously stirring and separating aqueous layers. The organic layer dried (MgSO₄), filtered, concentrated and purified by flash chromatography using 10, 20 and 25% EtOAc/Hex to afford desired (S)-ethyl 2-(5-bromo-4-(4,4-dimethylpiperidin-1-yl)-2,6-dimethylpyridin-3-yl)-2-hydroxyacetate (2.2596 g, 5.66 mmol, 100% yield) contaminated with about 10% of (S)-ethyl 2-(5-bromo-4-chloro-2,6-dimethylpyridin-3-yl)-2-hydroxyacetate. Used in the next step without further purification. ¹H NMR (500 MHz, CDCl₃) δ 5.71 (d, J=7.3 Hz, 1H), 5.54 (d, J=7.4 Hz, 1H), 4.29 (dq, J=10.8, 7.1 Hz, 1H), 4.16 (dq, J=10.8, 7.1 Hz, 1H), 3.94-3.83 (m, 2H), 2.71 (d, J=11.9 Hz, 1H), 2.67 (s, 3H), 2.59 (s, 3H), 2.54 (d, J=12.0 Hz, 1H), 1.71 (td, J=12.7, 4.7 Hz, 1H), 1.62 (td, J=13.0, 4.7 Hz, 1H), 1.42 (dd, J=13.1, 2.2 Hz, 1H), 1.37 (dd, J=12.9, 2.4 Hz, 1H), 1.25 (t, J=7.1 Hz, 3H), 1.09 (s, 3H), 1.04 (s, 3H). LCMS (M+H)=401.3.

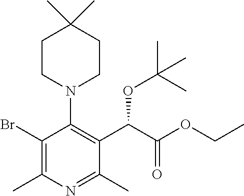 [SMILES: CCOC(=O)[C@@H](OC(C)(C)C)c1c(C)nc(C)c(Br)c1N1CCC(C)(C)CC1]

### (S)-Ethyl 2-(5-bromo-4-(4,4-dimethylpiperidin-1-yl)-2,6-dimethylpyridin-3-yl)-2-(tert butoxy)acetate

A stirred ice-cold yellow mixture of (S)-ethyl 2-(5-bromo-4-(4,4-dimethylpiperidin-1-yl)-2,6-dimethylpyridin-3-yl)-2-hydroxyacetate (2.45 g, 6.14 mmol) and 70% HClO₄ (1.054 ml, 12.27 mmol) in CH₂Cl₂ (100 mL) was saturated with isobutylene gas by bubbling through the reaction mixture (10 min). After 2 h, cold bath was removed and the turbid reaction mixture stirred for 22 h at rt. LCMS at this point showed 4:1 product to sm. So, saturated with isobutylene (5 min) at rt and stirred for additional 24 h. Then, neutralized with sat. Na₂CO₃ (30 mL), organic layer separated and aqueous layer extracted with CH₂Cl₂ (25 mL). The combined organic layers dried (MgSO₄), filtered, concentrated and purified by flash chromatography using 5, 10, 15, 20 and 40% EtOAc/hex to afford (S)-ethyl 2-(5-bromo-4-(4,4-dimethylpiperidin-1-yl)-2,6-dimethylpyridin-3-yl)-2-(tert-butoxy)acetate (2.3074 g, 5.07 mmol, 83% yield) as yellow oil: ¹H NMR (500 MHz, CDCl₃) δ 6.19 (br. s., 1H), 4.17-4.24 (m, 1H), 4.08-4.14 (m, 1H), 4.04 (dt, J=2.5, 12.1 Hz, 1H), 3.51 (dt, J=2.5, 12.1 Hz, 1H), 2.85-2.91 (m, 1H), 2.64 (s, 3H), 2.57-2.62 (m, 1H), 2.55 (s, 3H), 1.55-1.66 (m, 2H), 1.41-1.46 (m, 1H), 1.32-1.37 (m, 1H), 1.21 (s, 9H), 1.20 (t, J=7.2 Hz, 2H), 1.08 (s, 3H), 1.03 (s, 3H). LCMS (M+H)=457.4. And (S)-ethyl 2-(5-bromo-4-(4,4-dimethylpiperidin-1-yl)-2,6-dimethylpyridin-3-yl)-2-hydroxyacetate (0.3 g, 0.751 mmol, 12.24% yield) as pale yellow paste: LCMS (M+H)=401.3.

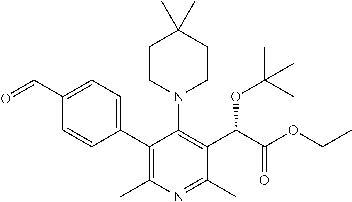 [SMILES: CCOC(=O)[C@@H](OC(C)(C)C)c1c(C)nc(C)c(-c2ccc(C=O)cc2)c1N1CCC(C)(C)CC1]

### (S)-Ethyl 2-(tert-butoxy)-2-(4-(4,4-dimethylpiperidin-1-yl)-5-(4-formylphenyl)-2,6-dimethylpyridin-3-yl)acetate

A mixture of (S)-ethyl 2-(5-bromo-4-(4,4-dimethylpiperidin-1-yl)-2,6-dimethylpyridin-3-yl)-2-(tert-butoxy)acetate (0.505 g, 1.109 mmol), (4-formylphenyl)boronic acid (0.333 g, 2.218 mmol) and 2M Na₂CO₃ (1.663 ml, 3.33 mmol) in DMF (10 mL) was degassed for 10 min. Then, Pd(Ph₃P)₄ (0.064 g, 0.055 mmol) was added, degassed for 5 min and placed in a pre-heated oilbath at 110° C. After 2 h, cooled, diluted with ether (50 mL), washed with water (4×10 mL), brine (10 mL), dried (MgSO₄), filtered, concentrated and purified by flash chromatography using 20, 30 and 40% EtOAc/Hex to afford (S)-ethyl 2-(tert-butoxy)-2-(4-(4,4-dimethylpiperidin-1-yl)-5-(4-formylphenyl)-2,6-dimethylpyridin-3-yl)acetate (0.426 g, 0.886 mmol, 80% yield) as off-white solid. ¹H NMR (500 MHz, CDCl₃) δ 10.13 (s, 1H), 8.00 (dt, J=1.4, 8.6 Hz, 2H), 7.49-7.53 (m, 1H), 7.38 (dd, J=1.3, 7.6 Hz, 1H), 6.03 (s, 1H), 4.24-4.31 (m, 1H), 4.16-4.24 (m, 1H), 3.26 (d, J=12.0 Hz, 1H), 2.85 (t, J=12.1 Hz, 1H), 2.63 (s, 3H), 2.26-2.33 (m, 1H), 2.19 (s, 3H), 1.94 (t, J=11.4 Hz, 1H), 1.56 (dt, J=3.6, 12.9 Hz, 1H), 1.32-1.42 (m, 1H), 1.28 (t, J=7.1 Hz, 3H), 1.21 (s, 9H), 1.02-1.08 (m, 1H), 0.90 (br. s., 3H), 0.60 (s, 3H). LCMS (M+H)=481.3.

### Example 1

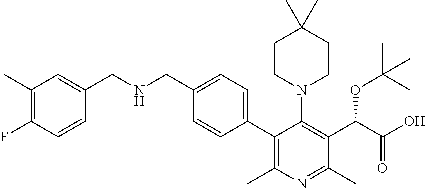 [SMILES: Cc1cc(CNCc2ccc(-c3c(C)nc(C)c([C@H](OC(C)(C)C)C(=O)O)c3N3CCC(C)(C)CC3)cc2)ccc1F]

(S)-2-(tert-Butoxy)-2-(4-(4,4-dimethylpiperidin-1-yl)-5-(4-(((4-fluoro-3-methylbenzyl)amino)methyl)phenyl)-2,6-dimethylpyridin-3-yl)acetic acid:

To a stirred solution of (S)-ethyl 2-(tert-butoxy)-2-(4-(4,4-dimethylpiperidin-1-yl)-5-(4-formylphenyl)-2,6-dimethylpyridin-3-yl)acetate (0.062 g, 0.052 mmol) and (4-fluoro-3-methylphenyl)methanamine (0.043 g, 0.310 mmol) in MeOH (5 mL) was added at once a mixture of ZnCl₂ (7.03 mg, 0.052 mmol) and NaCNBH₄ (6.49 mg, 0.103 mmol) in MeOH (1 mL) at rt. After 2 h, diluted with EtOAc (25 mL), washed with water (2×5 mL), brine (5 mL), dried (MgSO₄), filtered and concentrated to give crude (S)-ethyl 2-(tert-butoxy)-2-(4-(4,4-dimethylpiperidin-1-yl)-5-(4-(((4-fluoro-3-methylbenzyl)amino)methyl)phenyl)-2,6-dimethylpyridin-3-yl)acetate which was used in the next step without purification. LCMS (M+H)=604.5.

A solution of above crude ester and LiOH (0.012 g, 0.516 mmol) in 9:1 EtOH/H₂O (2 mL) was heated at reflux for 3.5 h. Then, cooled and purified by prep-HPLC to afford (S)-2-(tert-butoxy)-2-(4-(4,4-dimethylpiperidin-1-yl)-5-(4-(((4-fluoro-3-methylbenzyl)amino)methyl)phenyl)-2,6-dimethylpyridin-3-yl)acetic acid.NH₄OAc (0.0225 g, 0.034 mmol, 66.8% yield) as white solid. ¹H NMR (500 MHz, CDCL₃) δ 7.44 (t, J=8.5 Hz, 2H), 7.26 (dd, J=1.6, 7.7 Hz, 1H), 7.19 (dd, J=1.7, 7.3 Hz, 1H), 7.11-7.16 (m, 2H), 6.99 (t, J=8.5 Hz, 1H), 5.96 (br. s., 1H), 3.92 (s, 2H), 3.79 (s, 2H), 2.71-2.97 (m, 9H), 2.67 (s, 3H), 2.30 (d, J=1.6 Hz, 3H), 2.23 (s, 3H), 2.10 (s, 3H), 1.26-1.35 (m, 4H), 1.25 (s, 9H), 0.74 (br. s., 6H). LCMS (M+H)=576.5.

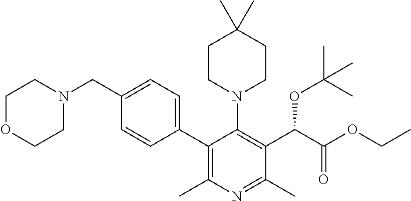 [SMILES: CCOC(=O)[C@@H](OC(C)(C)C)c1c(C)nc(C)c(-c2ccc(CN3CCOCC3)cc2)c1N1CCC(C)(C)CC1]

### (S)-Ethyl 2-(tert-butoxy)-2-(4-(4,4-dimethylpiperidin-1-yl)-2,6-dimethyl-5-(4-(morpholinomethyl)phenyl)pyridin-3-yl)acetate

A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-(morpholinomethyl)phenyl)boronic acid (0.019 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-2,6-dimethyl-5-(4-(morpholinomethyl)phenyl)pyridin-3-yl)acetate (0.0114 g, 0.021 mmol, 47.0% yield) as brown solid. LCMS (M+H)=552.5.

### Example 2

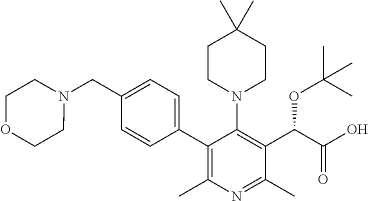 [SMILES: Cc1nc(C)c([C@H](OC(C)(C)C)C(=O)O)c(N2CCC(C)(C)CC2)c1-c1ccc(CN2CCOCC2)cc1]

### (S)-2-(tert-Butoxy)-2-(4-(4,4-dimethylpiperidin-1-yl)-2,6-dimethyl-5-(4-(morpholinomethyl)phenyl)pyridin-3-yl)acetic acid

A solution of (S)-ethyl 2-(tert-butoxy)-2-(4-(4,4-dimethylpiperidin-1-yl)-2,6-dimethyl-5-(4-(morpholinomethyl)phenyl)pyridin-3-yl)acetate (0.0114 g, 0.021 mmol) and 1M NaOH (0.207 ml, 0.207 mmol) in EtOH (1 mL) was refluxed for 6 h. Then, cooled and purified by prep-HPLC to afford (S)-2-(tert-butoxy)-2-(4-(4,4-dimethylpiperidin-1-yl)-2,6-dimethyl-5-(4-(morpholinomethyl)phenyl)pyridin-3-yl)acetic acid (0.0095 g, 0.018 mmol, 88% yield) as solid. ¹H NMR (500 MHz, DMSO-d₆) δ 7.43 (d, J=7.7 Hz, 1H), 7.38 (d, J=7.7 Hz, 1H), 7.29 (d, J=7.7 Hz, 1H), 7.11 (d, J=7.3 Hz, 1H), 5.87 (br. s., 1H), 3.60-3.49 (m, 6H), 3.22 (d, J=12.1 Hz, 1H), 2.79 (t, J=11.9 Hz, 1H), 2.45 (s, 3H), 2.37 (br. s., 4H), 2.17 (d, J=11.4 Hz, 1H), 2.07 (s, 3H), 1.82 (t, J=11.9 Hz, 1H), 1.52-1.42 (m, 1H), 1.19-1.14 (m, 1H), 1.13 (s, 9H), 0.96 (d, J=11.7 Hz, 1H), 0.83 (s, 3H), 0.52 (s, 3H). LCMS(M+H)=524.20.

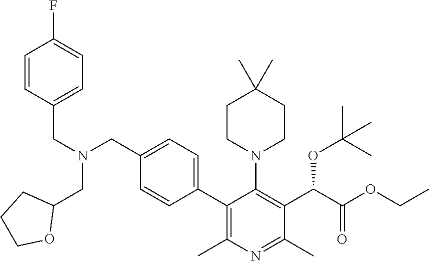 [SMILES: CCOC(=O)[C@@H](OC(C)(C)C)c1c(C)nc(C)c(-c2ccc(CN(Cc3ccc(F)cc3)CC3CCCO3)cc2)c1N1CCC(C)(C)CC1]

### (2S)-Ethyl 2-(tert-butoxy)-2-(4-(4,4-dimethylpiperidin-1-yl)-5-(4-(((4-fluorobenzyl) ((tetrahydrofuran-2-yl)methyl)amino)methyl)phenyl)-2,6-dimethylpyridin-3-yl)acetate

To a 5-mL RB flask was charged with (S)-ethyl 2-(tert-butoxy)-2-(4-(4,4-dimethylpiperidin-1-yl)-5-(4-formylphenyl)-2,6-dimethylpyridin-3-yl)acetate (0.02 g, 0.042 mmol), N-(4-fluorobenzyl)-1-(tetrahydrofuran-2-yl)methanamine, HCl (0.020 g, 0.083 mmol), NaCNBH4 (5.23 mg, 0.083 mmol) and ZnCl2 (2.84 mg, 0.021 mmol) was added MeOH (1 ML) and a drop of Et3N. The resulting clear reaction mixture was stirred at rt for 24 h. LCMS at this point showed completion of reaction. Diluted with EtOAc (25 mL), washed with sat Na2CO3 (5 mL), water (5 mL), brine (5 mL), dried (MgSO4), filtered and concentrated to give (2S)-ethyl 2-(tert-butoxy)-2-(4-(4,4-dimethylpiperidin-1-yl)-5-(4-(((4-fluorobenzyl)((tetrahydrofuran-2-yl)methyl)amino)methyl)phenyl)-2,6-dimethylpyridin-3-yl)acetate as paste which was used in the next step without purification. LCMS (M+H)=674.8.

### Example 3

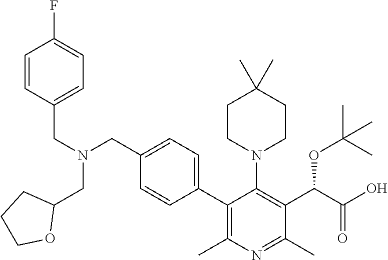 [SMILES: Cc1nc(C)c([C@H](OC(C)(C)C)C(=O)O)c(N2CCC(C)(C)CC2)c1-c1ccc(CN(Cc2ccc(F)cc2)CC2CCCO2)cc1]

### (2S)-2-(tert-Butoxy)-2-(4-(4,4-dimethylpiperidin-1-yl)-5-(4-(((4-fluorobenzyl) ((tetrahydrofuran-2-yl)methyl)amino)methyl)phenyl)-2,6-dimethylpyridin-3-yl)acetic acid

A solution of (2S)-ethyl 2-(tert-butoxy)-2-(4-(4,4-dimethylpiperidin-1-yl)-5-(4-(((4-fluorobenzyl)((tetrahydrofuran-2-yl)methyl)amino)methyl)phenyl)-2,6-dimethylpyridin-3-yl)acetate (0.028 g, 0.042 mmol) and 1M NaOH (0.210 ml, 0.210 mmol) in EtOH was refluxed for 8 h. Then, cooled and purified by prep-HPLC to afford (2 S)-2-(tert-butoxy)-2-(4-(4,4-dimethylpiperidin-1-yl)-5-(4-(((4-fluorobenzyl)((tetrahydrofuran-2-yl)methyl)amino)methyl)phenyl)-2,6-dimethylpyridin-3-yl)acetic acid (0.0177 g, 0.027 mmol, 65.3% yield) as solid and mixture of diastereomers. ¹H NMR (500 MHz, DMSO-d₆) δ 7.48-7.34 (m, 4H), 7.29-7.25 (m, 1H), 7.13 (t, J=8.6 Hz, 2H), 7.09 (t, J=5.9 Hz, 1H), 5.75 (s, 1H), 4.01 (quin, J=6.1 Hz, 1H), 3.75 (dd, J=13.6, 5.5 Hz, 1H), 3.66 (s, 1H), 3.62-3.55 (m, 2H), 3.52 (dd, J=13.9, 4.8 Hz, 1H), 3.44-3.37 (m, 1H), 2.81-2.73 (m, 1H), 2.53-2.40 (m, 2H), 2.44 (s, 3H), 2.19-2.12 (m, 1H), 2.05 (s, 1.5H), 2.04 (s, 1.5H), 1.88-1.78 (m, 2H), 1.75-1.64 (m, 2H), 1.50-1.33 (m, 2H), 1.24 (d, J=8.4 Hz, 1H), 1.11 (s, 9H), 0.86 (br. s., 1H), 0.77 (br. s., 3H), 0.42 (br. s., 3H). 2H of piperidine were not resolved. LCMS (M+H)=646.25.

### Synthesis of (S)-2-(5-(4-(N-substituted aminomethyl)phenyl)-4-(4,4-dimethylpiperidin-1-yl)-2,6-dimethylpyridin-3-yl)-2-(tert-butoxy)acetic acid from (S)-ethyl 2-(5-bromo-4-(4,4-dimethylpiperidin-1-yl)-2,6-dimethylpyridin-3-yl)-2-(tert-butoxy)acetate

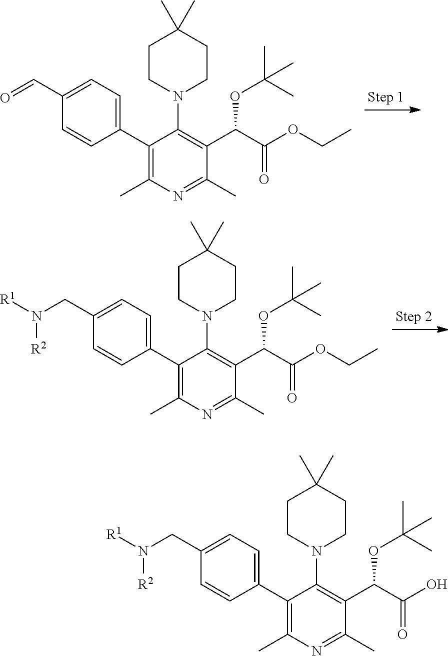

Step 1: General procedure: ZnCl₂ (0.5 eq.) and NaCNBH₃ (2 eq.) were added into a solution of (S)-ethyl 2-(tert-butoxy)-2-(4-(4,4-dimethylpiperidin-1-yl)-5-(4-formylphenyl)-2,6-dimethylpyridin-3-yl)acetate (1 eq.) and amine (1 eq.) in methanol. The reaction mixture was stirred at room temperature 16 hours. The desired ester was isolated by the preparative HPLC system.

| | | | LCMS |
|---|---|---|---|
| | | | (M + |
| Name | HNR¹R² | Structure | H) |
| (S)-ethyl 2-(tert- butoxy)-2-(4- (4,4-dimethyl- piperidin- 1-yl)-5-(4-(((4- fluorobenzyl) amino)methyl) phenyl)-2,6- dimethyl- pyridin-3-yl) acetate | 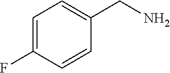 | 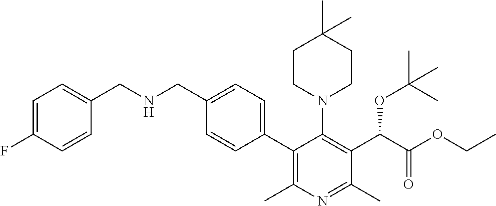 [SMILES: CCOC(=O)[C@@H](OC(C)(C)C)c1c(C)nc(C)c(-c2ccc(CNCc3ccc(F)cc3)cc2)c1N1CCC(C)(C)CC1] | 590.4 |
| (S)-ethyl 2-(tert- butoxy)- 2-(4-(4,4- dimethyl- piperidin-1-yl)- 5-(4-(((4-fluoro- phenethyl) (methyl)amino) methyl)phenyl)- 2,6-dimeth- ylpyridin- 3-yl)acetate | 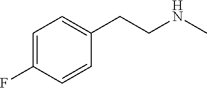 | 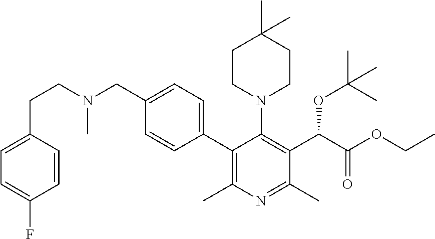 [SMILES: CCOC(=O)[C@@H](OC(C)(C)C)c1c(C)nc(C)c(-c2ccc(CN(C)CCc3ccc(F)cc3)cc2)c1N1CCC(C)(C)CC1] | 618.3 |
| (S)-ethyl 2-(tert- butoxy)- 2-(5-(4-(((3,3- dimethylbutyl) amino)methyl) phenyl)-4-(4,4- dimethyl- piperidin- 1-yl)-2,6- dimethyl- pyridin- 3-yl)acetate | 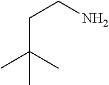 | 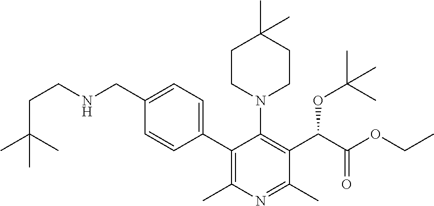 [SMILES: CCOC(=O)[C@@H](OC(C)(C)C)c1c(C)nc(C)c(-c2ccc(CNCCC(C)(C)C)cc2)c1N1CCC(C)(C)CC1] | 566.5 |
| (S)-ethyl 2- (tert-butoxy)- 2-(4-(4,4- dimethyl- piperidin- 1-yl)-5-(4-(((4- fluorobenzyl) (methyl)amino) methyl) phenyl)- 2,6-dimethyl- pyridin-3-yl) acetate | 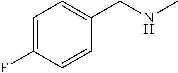 | 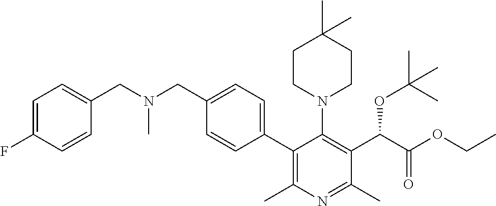 [SMILES: CCOC(=O)[C@@H](OC(C)(C)C)c1c(C)nc(C)c(-c2ccc(CN(C)Cc3ccc(F)cc3)cc2)c1N1CCC(C)(C)CC1] | 604.3 |
| (S)-ethyl 2-(tert- butoxy)-2- (4-(4,4- dimethyl- piperidin- 1-yl)-5- (4-(((4- methoxy- phenethyl) amino) methyl)- phenyl)-2,6- dimethyl- pyridin-3-yl) | 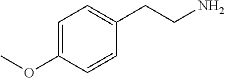 | 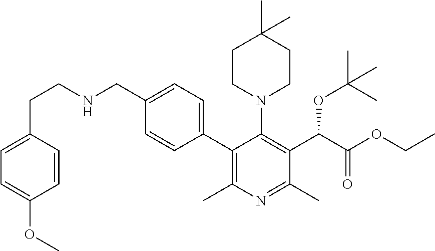 [SMILES: CCOC(=O)[C@@H](OC(C)(C)C)c1c(C)nc(C)c(-c2ccc(CNCCc3ccc(OC)cc3)cc2)c1N1CCC(C)(C)CC1] | 616.4 |
| acetate | | | |
| (S)-ethyl 2-(tert- butoxy)- 2-(4-(4,4- dimethyl- piperidin-1- yl)-5-(4- (((2-methoxy- phenethyl) amino)meth- yl)phenyl)- 2,6-dimethyl- pyridin-3-yl) | 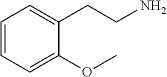 | 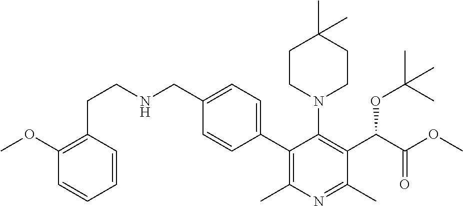 | 616.4 |
| acetate | | | |
| (S)-ethyl 2- (tert-butoxy)- 2-(4-(4,4- dimethyl- piperidin-1- yl)-5- (4-(((4-fluoro- benzyl) (methoxy) amino)methyl) phenyl)-2,6- dimethyl- pyridin- | 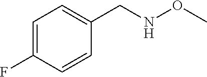 | 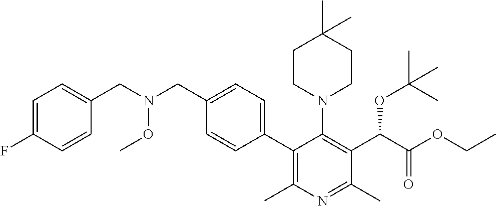 [SMILES: CCOC(=O)[C@@H](OC(C)(C)C)c1c(C)nc(C)c(-c2ccc(CN(Cc3ccc(F)cc3)OC)cc2)c1N1CCC(C)(C)CC1] | 620.3 |
| 3-yl)acetate | | | |
| (S)-ethyl 2-(tert- butoxy)- 2-(4-(4,4- dimethyl- piperidin- 1-yl)-5-(4- (((4-fluoro- phenethyl) amino)methyl) phenyl)- 2,6-dimethyl- pyridin-3-yl) acetate | 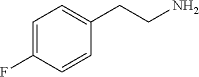 | 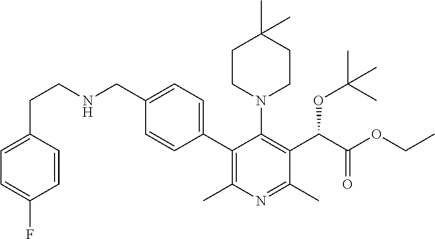 [SMILES: CCOC(=O)[C@@H](OC(C)(C)C)c1c(C)nc(C)c(-c2ccc(CNCCc3ccc(F)cc3)cc2)c1N1CCC(C)(C)CC1] | 604.3 |
| (S)-ethyl 2- (tert-butoxy)- 2-(5-(4-(((3,4- dichlorobenzyl) amino)methyl) phenyl)-4-(4,4- dimethyl- piperidin- 1-yl)-2,6- dimethyl- pyridin- 3-yl)acetate | 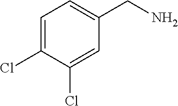 | 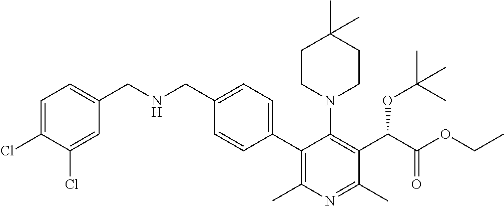 [SMILES: CCOC(=O)[C@@H](OC(C)(C)C)c1c(C)nc(C)c(-c2ccc(CNCc3ccc(Cl)c(Cl)c3)cc2)c1N1CCC(C)(C)CC1] | 640.2 |
| (S)-ethyl 2-(tert- butoxy)- 2-(5-(4-(((2- cyclo- hexylethyl) amino)methyl) phenyl)-4-(4,4- dimethyl- piperidin- 1-yl)-2,6-di- methylpyridin- 3-yl)acetate | 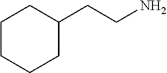 | 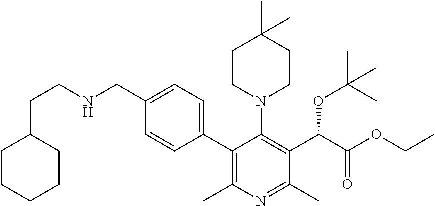 [SMILES: CCOC(=O)[C@@H](OC(C)(C)C)c1c(C)nc(C)c(-c2ccc(CNCCC3CCCCC3)cc2)c1N1CCC(C)(C)CC1] | 592.5 |
| (S)-ethyl 2-(5-(4- ((benzylamino) methyl) phenyl)- 4-(4,4- dimethyl- piperidin-1-yl)- 2,6-di- methylpyridin- 3-yl)-2- (tert-butoxy) acetate | 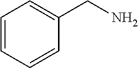 | 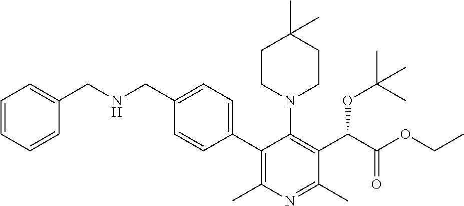 [SMILES: CCOC(=O)[C@@H](OC(C)(C)C)c1c(C)nc(C)c(-c2ccc(CNCc3ccccc3)cc2)c1N1CCC(C)(C)CC1] | 572.3 |
| (S)-ethyl 2- (tert-butoxy)- 2-(5-(4-(((4- chlorobenzyl) amino)methyl) phenyl)-4-(4,4- dimethyl- piperidin- 1-yl)-2,6- dimethyl- pyridin- 3-yl)acetate | 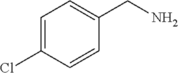 | 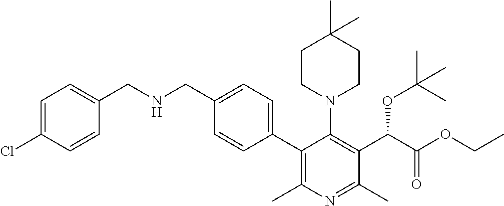 [SMILES: CCOC(=O)[C@@H](OC(C)(C)C)c1c(C)nc(C)c(-c2ccc(CNCc3ccc(Cl)cc3)cc2)c1N1CCC(C)(C)CC1] | 606.3 |
| (S)-ethyl 2-(tert- butoxy)-2-(4- (4,4-dimethyl- piperidin- 1-yl)-2,6- dimethyl- 5-(4-(((4- methylbenzyl) amino) methyl)phenyl) pyridin-3-yl) acetate | 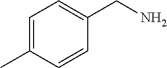 | 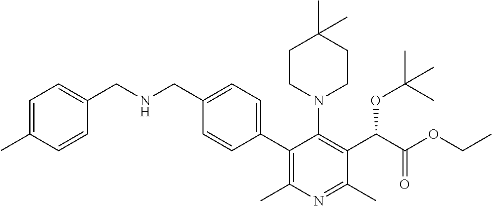 [SMILES: CCOC(=O)[C@@H](OC(C)(C)C)c1c(C)nc(C)c(-c2ccc(CNCc3ccc(C)cc3)cc2)c1N1CCC(C)(C)CC1] | 586.4 |
| (S)-ethyl 2-(tert- butoxy)- 2-(5-(4- (((cyclohexyl- methyl) amino)methyl) phenyl)- 4-(4,4-di- methyl- piperidin- 1-yl)-2,6- dimethyl- | 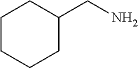 | 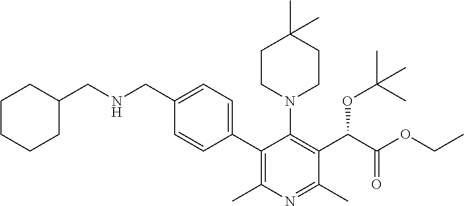 [SMILES: CCOC(=O)[C@@H](OC(C)(C)C)c1c(C)nc(C)c(-c2ccc(CNCC3CCCCC3)cc2)c1N1CCC(C)(C)CC1] | 578.3 |
| pyridin- | | | |
| 3-yl)acetate | | | |

Step 2: General procedure: NaOH (3 eq.) was added to a solution of the ester obtained in the step 1 (1 eq.) in EtOH or MeOH and water (volume ratio 1:1). The reaction was heated at 85° C. for 1-2 h. The desired acid was isolated by the preparative HPLC system.

| | | LCMS |
|---|---|---|
| Name | Structure | (M + H) |
| (S)-2-(tert-butoxy)-2-(4- (4,4-dimethylpiperidin-1- yl)-5-(4-(((4- fluorobenzyl)amino)methyl) phenyl)-2,6- dimethylpyridin-3-yl)acetic acid | 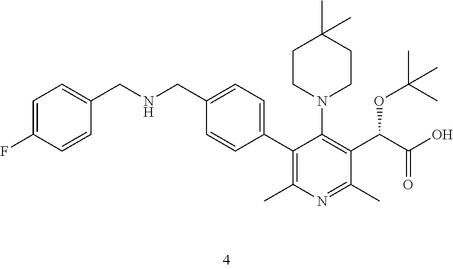 [SMILES: Cc1nc(C)c([C@H](OC(C)(C)C)C(=O)O)c(N2CCC(C)(C)CC2)c1-c1ccc(CNCc2ccc(F)cc2)cc1] | 562.2 |
| (S)-2-(tert-butoxy)-2-(4- (4,4-dimethylpiperidin-1- yl)-5-(4-(((4- fluorophenethyl)(methyl) amino)methyl)phenyl)-2,6- dimethylpyridin-3-yl)acetic acid | 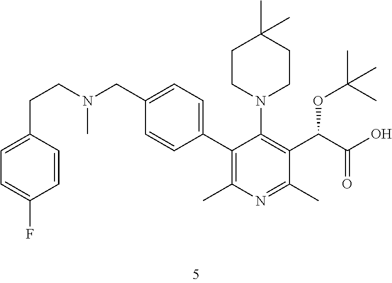 [SMILES: Cc1nc(C)c([C@H](OC(C)(C)C)C(=O)O)c(N2CCC(C)(C)CC2)c1-c1ccc(CN(C)CCc2ccc(F)cc2)cc1] | 590.3 |
| (S)-2-(tert-butoxy)-2-(5-(4- (((3,3- dimethylbutyl)amino)meth- yl)phenyl)-4-(4,4- dimethylpiperidin-1-yl)- 2,6-dimethylpyridin-3- yl)acetic acid | 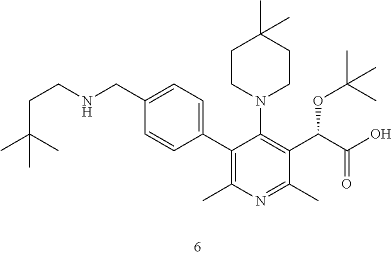 [SMILES: Cc1nc(C)c([C@H](OC(C)(C)C)C(=O)O)c(N2CCC(C)(C)CC2)c1-c1ccc(CNCCC(C)(C)C)cc1] | 538.5 |
| (S)-2-(tert-butoxy)-2-(4- (4,4-dimethylpiperidin-1- yl)-5-(4-(((4- fluorobenzyl)(methyl)amino) methyl)phenyl)-2,6- dimethylpyridin-3-yl)acetic acid | 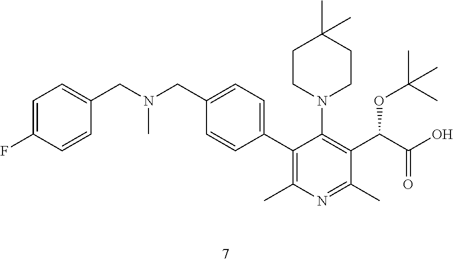 [SMILES: Cc1nc(C)c([C@H](OC(C)(C)C)C(=O)O)c(N2CCC(C)(C)CC2)c1-c1ccc(CN(C)Cc2ccc(F)cc2)cc1] | 576.3 |
| (S)-2-(tert-butoxy)-2-(4- (4,4-dimethylpiperidin-1- yl)-5-(4-(((4- methoxyphenethyl)amino) methyl)phenyl)-2,6- dimethylpyridin-3-yl)acetic acid | 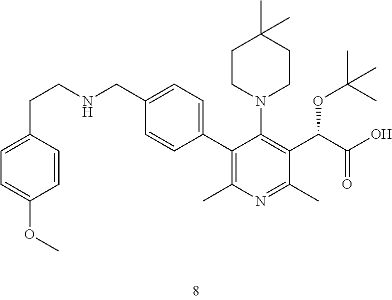 [SMILES: COc1ccc(CCNCc2ccc(-c3c(C)nc(C)c([C@H](OC(C)(C)C)C(=O)O)c3N3CCC(C)(C)CC3)cc2)cc1] | 588.4 |
| (S)-2-(tert-butoxy)-2-(4- (4,4-dimethylpiperidin-1- yl)-5-(4-(((2- methoxyphenethyl)amino) methyl)phenyl)-2,6- dimethylpyridin-3-yl)acetic acid | 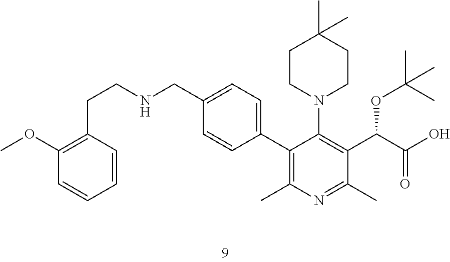 [SMILES: COc1ccccc1CCNCc1ccc(-c2c(C)nc(C)c([C@H](OC(C)(C)C)C(=O)O)c2N2CCC(C)(C)CC2)cc1] | 588.4 |
| (S)-2-(tert-butoxy)-2-(4- (4,4-dimethylpiperidin-1- yl)-5-(4-(((4- fluorobenzyl)(methoxy) amino)methyl)phenyl)-2,6- dimethylpyridin-3-yl)acetic acid | 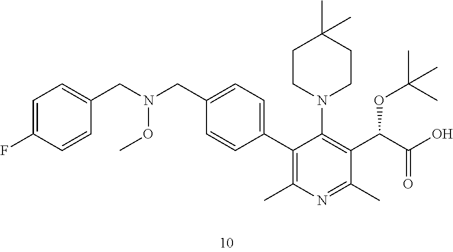 [SMILES: CON(Cc1ccc(F)cc1)Cc1ccc(-c2c(C)nc(C)c([C@H](OC(C)(C)C)C(=O)O)c2N2CCC(C)(C)CC2)cc1] | 592.3 |
| (S)-2-(tert-butoxy)-2-(4- (4,4-dimethylpiperidin-1- yl)-5-(4-(((4- fluorophenethyl)amino)meth- yl)phenyl)-2,6- dimethylpyridin-3-yl)acetic acid | 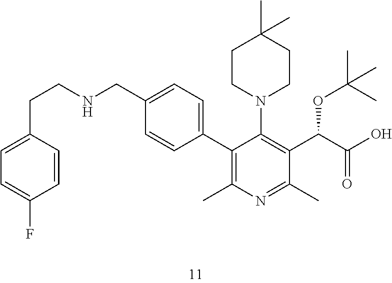 [SMILES: Cc1nc(C)c([C@H](OC(C)(C)C)C(=O)O)c(N2CCC(C)(C)CC2)c1-c1ccc(CNCCc2ccc(F)cc2)cc1] | 576.3 |
| (S)-2-(tert-butoxy)-2-(5-(4- (((3,4- dichlorobenzyl)amino)meth- yl)phenyl)-4-(4,4- dimethylpiperidin-1-yl)- 2,6-dimethylpyridin-3- yl)acetic acid | 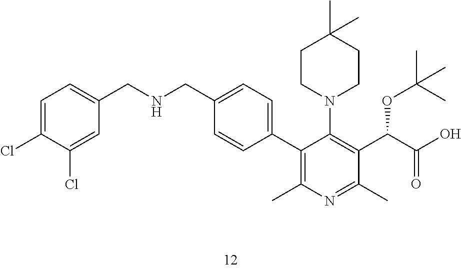 [SMILES: Cc1nc(C)c([C@H](OC(C)(C)C)C(=O)O)c(N2CCC(C)(C)CC2)c1-c1ccc(CNCc2ccc(Cl)c(Cl)c2)cc1] | 612.2 |
| (S)-2-(tert-butoxy)-2-(5-(4- (((2- cyclohexylethyl)amino)meth- yl)phenyl)-4-(4,4- dimethylpiperidin-1-yl)- 2,6-dimethylpyridin-3- yl)acetic acid | 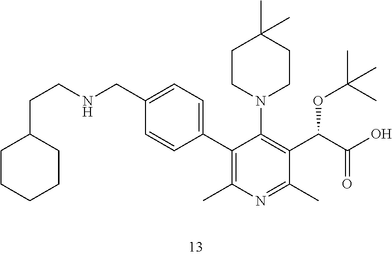 [SMILES: Cc1nc(C)c([C@H](OC(C)(C)C)C(=O)O)c(N2CCC(C)(C)CC2)c1-c1ccc(CNCCC2CCCCC2)cc1] | 564.3 |
| (S)-2-(5-(4- ((benzylamino)methyl) phenyl)-4-(4,4- dimethylpiperidin-1-yl)- 2,6-dimethylpyridin-3-yl)- 2-(tert-butoxy)acetic acid | 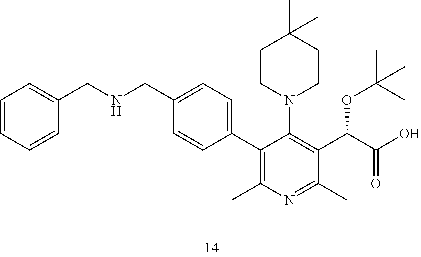 [SMILES: Cc1nc(C)c([C@H](OC(C)(C)C)C(=O)O)c(N2CCC(C)(C)CC2)c1-c1ccc(CNCc2ccccc2)cc1] | 544.5 |
| (S)-2-(tert-butoxy)-2-(5-(4- (((4- chlorobenzyl)amino)methyl) phenyl)-4-(4,4- dimethylpiperidin-1-yl)- 2,6-dimethylpyridin-3- yl)acetic acid | 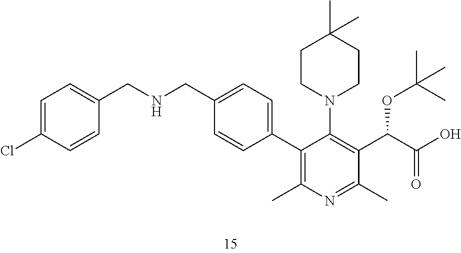 [SMILES: Cc1nc(C)c([C@H](OC(C)(C)C)C(=O)O)c(N2CCC(C)(C)CC2)c1-c1ccc(CNCc2ccc(Cl)cc2)cc1] | 578.3 |
| (S)-2-(tert-butoxy)-2-(4- (4,4-dimethylpiperidin-1- yl)-2,6-dimethyl-5-(4-(((4- methylbenzyl)amino)meth- yl)phenyl)pyridin-3-yl)acetic acid | 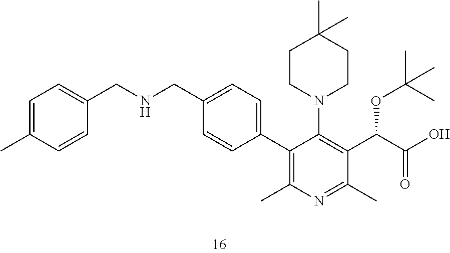 [SMILES: Cc1ccc(CNCc2ccc(-c3c(C)nc(C)c([C@H](OC(C)(C)C)C(=O)O)c3N3CCC(C)(C)CC3)cc2)cc1] | 558.2 |
| (S)-2-(tert-butoxy)-2-(5-(4- (((cyclohexylmethyl)amino) methyl)phenyl)-4-(4,4- dimethylpiperidin-1-yl)- 2,6-dimethylpyridin-3- yl)acetic acid | 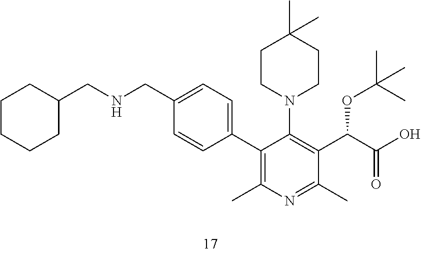 [SMILES: Cc1nc(C)c([C@H](OC(C)(C)C)C(=O)O)c(N2CCC(C)(C)CC2)c1-c1ccc(CNCC2CCCCC2)cc1] | 550.3 |

¹HNMR for example 7:

¹H NMR (500 MHz, CD₃OD) δ 7.78-7.26 (m, 8H), 5.70 (s, 1H), 4.45 (m, 4H), 2.84-2.78 (m, 10H), 2.61 (s, 3H), 1.37-1.25 (m, 13H), 0.84 (s, 6H).

### Synthesis of (S)-2-(tert-butoxy)-2-(4-(4,4-dimethylpiperidin-1-yl)-5-(4-(((4-(methoxycarbonyl)benzyl)amino)methyl)phenyl)-2,6-dimethylpyridin-3-yl)acetic acid, and, (S)-4-(((4-(5-(tert-butoxy(carboxy)methyl)-4-(4,4-dimethylpiperidin-1-yl)-2,6-dimethylpyridin-3-yl)benzyl)amino)methyl)benzoic acid

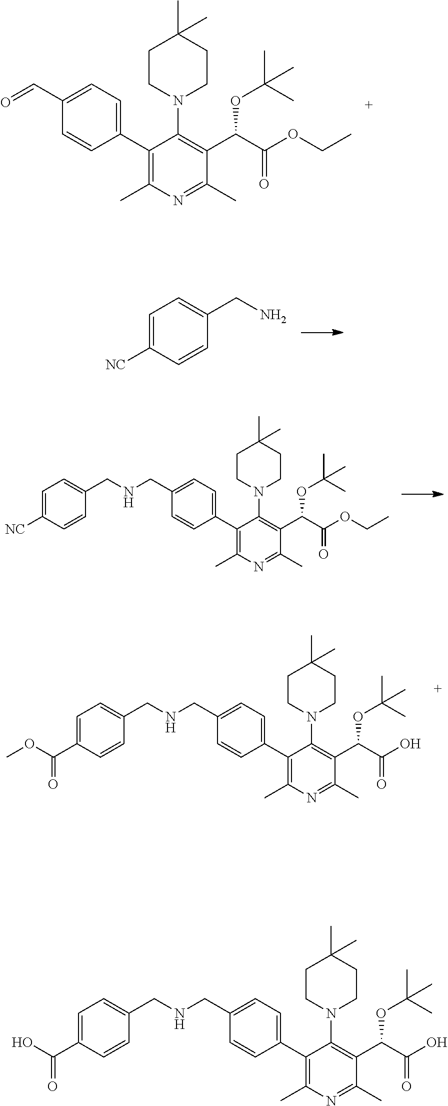

Step 1: ZnCl₂ (1.79 mg) and NaCHBH₃ (3.29 mg) were added to a solution of (S)-ethyl 2-(tert-butoxy)-2-(4-(4,4-dimethylpiperidin-1-yl)-5-(4-formylphenyl)-2,6-dimethylpyridin-3-yl)acetate (12.6 mg) and 4-(aminomethyl)benzonitrile (3.46 mg) in methanol (2 mL). The mixture was stirred at room temperature for 48 h before the product was isolated by the preparative HPLC. LCMS MS (M+H): 597.3.

Step 2: NaOH (3.02 mg) was added to a solution of (S)-ethyl 2-(tert-butoxy)-2-(5-(4-(((4-cyanobenzyl)amino)methyl)phenyl)-4-(4,4-dimethylpiperidin-1-yl)-2,6-dimethylpyridin-3-yl)acetate (15 mg) in methanol (2 mL) and water (0.2 mL). The reaction mixture was heated at 85° C. for 1 h before the products were isolated by the preparative HPLC.

| | | LCMS |
|---|---|---|
| Name | Structure | (M + H) |
| (S)-2-(tert-butoxy)-2-(4- (4,4-dimethylpiperidin- 1-yl)-5-(4-(((4- (methoxycarbonyl)benzyl) amino)methyl)phenyl)- 2,6-dimethylpyridin-3- yl)acetic acid | 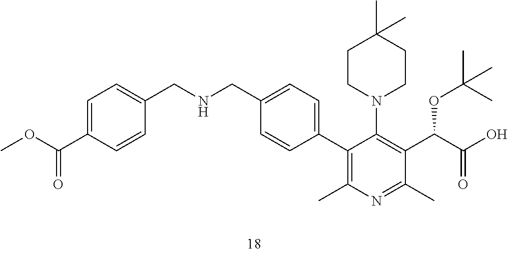 [SMILES: COC(=O)c1ccc(CNCc2ccc(-c3c(C)nc(C)c([C@H](OC(C)(C)C)C(=O)O)c3N3CCC(C)(C)CC3)cc2)cc1] | 602.4 |
| (S)-4-(((4-(5-(tert- butoxy(carboxy)methyl)- 4-(4,4- dimethylpiperidin-1-yl)- 2,6-dimethylpyridin-3- yl)benzyl)amino)methyl) benzoic acid | 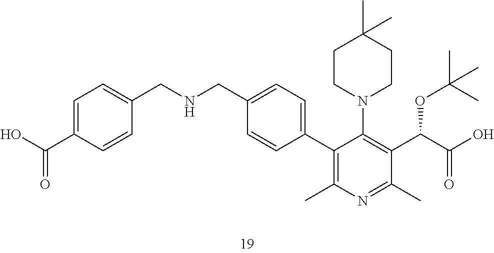 [SMILES: Cc1nc(C)c([C@H](OC(C)(C)C)C(=O)O)c(N2CCC(C)(C)CC2)c1-c1ccc(CNCc2ccc(C(=O)O)cc2)cc1] | 588.2 |

### Syntheses of (S)-2-(tert-butoxy)-2-(4-(4,4-dimethylpiperidin-1-yl)-5-(4-(((4-fluorobenzyl)substituted amino)methyl)phenyl)-2,6-dimethylpyridin-3-yl)acetic acid

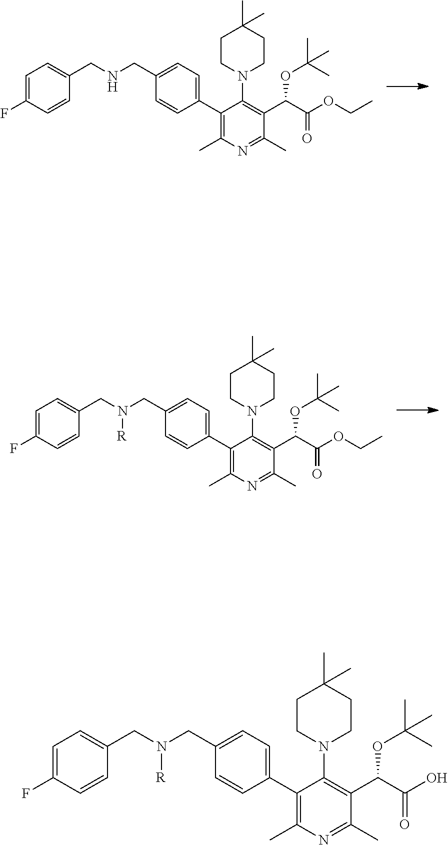

Step 1: General procedure: iPr₂NEt (2 eq.) and electrophile (1 eq.) were added to a solution of (S)-ethyl 2-(tert-butoxy)-2-(4-(4,4-dimethylpiperidin-1-yl)-5-(4-(((4-fluorobenzyl)amino)methyl)phenyl)-2,6-dimethylpyridin-3-yl)acetate (1 eq.) in THF. The reaction was stirred at room temperature for 2 hours. Solvents were removed under vacuum to give a crude product which was used as is or isolated by the preparative HPLC.

| | | | LCMS |
|---|---|---|---|
| | Electro- | | (M + |
| Name | phile | Structure | H) |
| (S)-ethyl 2-(tert- butoxy)-2-(4- (4,4-dimethylpiperidin- 1-yl)-5-(4-((N-(4- fluorobenzyl)cyclopentane- carboxamido)methyl) phenyl)- 2,6-dimethylpyridin-3- yl)acetate | 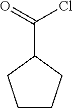 | 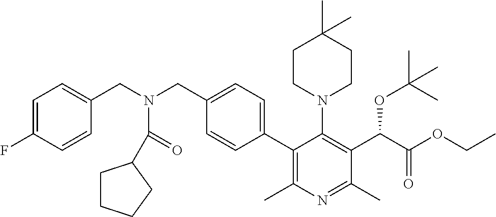 [SMILES: CCOC(=O)[C@@H](OC(C)(C)C)c1c(C)nc(C)c(-c2ccc(CN(Cc3ccc(F)cc3)C(=O)C3CCCC3)cc2)c1N1CCC(C)(C)CC1] | 686.5 |
| (S)-ethyl 2-(tert- butoxy)-2-(4- (4,4-dimethylpiperidin- 1-yl)-5-(4-((N-(4- fluorobenzyl)benzamido) methyl)phenyl)-2,6- dimethylpyridin-3-yl)acetate | 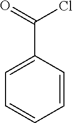 | 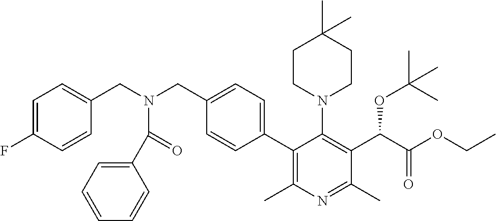 [SMILES: CCOC(=O)[C@@H](OC(C)(C)C)c1c(C)nc(C)c(-c2ccc(CN(Cc3ccc(F)cc3)C(=O)c3ccccc3)cc2)c1N1CCC(C)(C)CC1] | 694.5 |
| (S)-ethyl 2-(tert- butoxy)-2-(4- (4,4-dimethylpieridin-1-yl)- 5-(4-((N-(4- fluorobenzyl)propionamido) methyl)phenyl)-2,6- dimethylpyridin-3-yl)acetate | 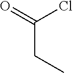 | 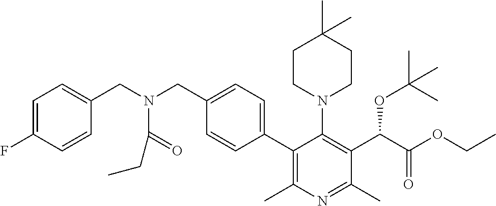 [SMILES: CCOC(=O)[C@@H](OC(C)(C)C)c1c(C)nc(C)c(-c2ccc(CN(Cc3ccc(F)cc3)C(=O)CC)cc2)c1N1CCC(C)(C)CC1] | 646.5 |
| (S)-ethyl 2-(tert- butoxy)-2-(4- (4,4-dimethylpiperidin- 1-yl)-5-(4-((N-(4- fluorobenzyl) isobutyramido) methyl)phenyl)-2,6- dimethylpyridin-3-yl) acetate | 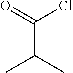 | 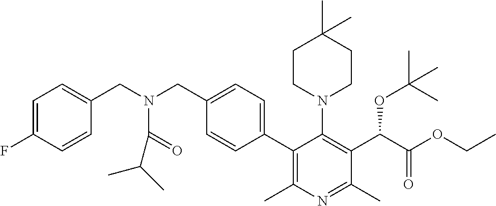 [SMILES: CCOC(=O)[C@@H](OC(C)(C)C)c1c(C)nc(C)c(-c2ccc(CN(Cc3ccc(F)cc3)C(=O)C(C)C)cc2)c1N1CCC(C)(C)CC1] | 660.5 |
| (S)-ethyl 2-(tert- butoxy)-2-(4- (4,4-dimethylpiperidin- 1-yl)-5-(4-((N-(4- fluorobenzyl)acetamido) methyl)phenyl)-2,6- dimethylpyridin-3-yl) acetate | 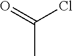 | 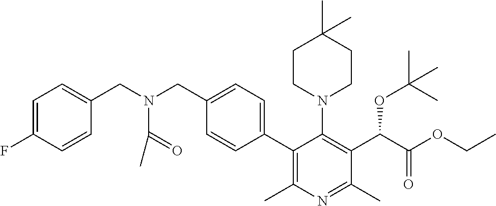 [SMILES: CCOC(=O)[C@@H](OC(C)(C)C)c1c(C)nc(C)c(-c2ccc(CN(Cc3ccc(F)cc3)C(C)=O)cc2)c1N1CCC(C)(C)CC1] | 632.5 |
| (S)-ethyl 2-(tert- butoxy)-2-(4- (4,4-dimethylpiperidin- 1-yl)-5-(4-((N-(4- fluorobenzyl)pivalamido) methyl)phenyl)-2,6- dimethylpyridin-3-yl) acetate | 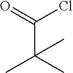 | 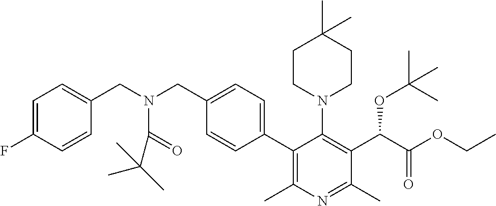 [SMILES: CCOC(=O)[C@@H](OC(C)(C)C)c1c(C)nc(C)c(-c2ccc(CN(Cc3ccc(F)cc3)C(=O)C(C)(C)C)cc2)c1N1CCC(C)(C)CC1] | 674.6 |
| (S)-ethyl 2-(tert- butoxy)-2-(4- (4,4-dimethylpiperidin- 1-yl)-5-(4-((N- (4-fluorobenzyl)-2- methoxybenzamido) methyl)phenyl)-2,6- dimethylpyridin- 3-yl)acetate | 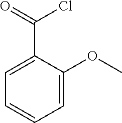 | 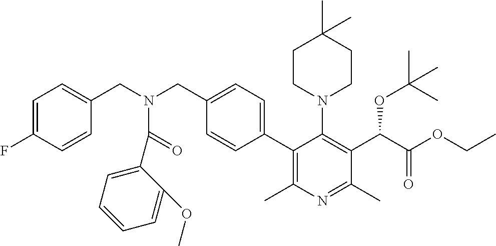 [SMILES: CCOC(=O)[C@@H](OC(C)(C)C)c1c(C)nc(C)c(-c2ccc(CN(Cc3ccc(F)cc3)C(=O)c3ccccc3OC)cc2)c1N1CCC(C)(C)CC1] | 724.6 |
| (S)-ethyl 2-(tert- butoxy)-2-(4- (4,4-dimethylpiperidin- 1-yl)- 5-(4-((2-fluoro-N-(4- fluorobenzyl)benzamido) methyl)phenyl)-2,6- dimethylpyridin-3-yl) acetate | 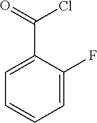 | 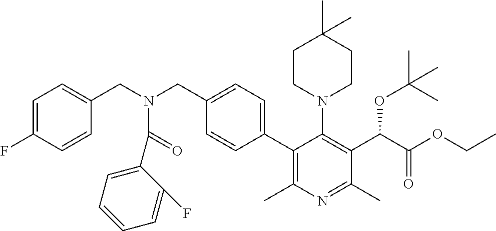 [SMILES: CCOC(=O)[C@@H](OC(C)(C)C)c1c(C)nc(C)c(-c2ccc(CN(Cc3ccc(F)cc3)C(=O)c3ccccc3F)cc2)c1N1CCC(C)(C)CC1] | 712.5 |
| (S)-ethyl 2-(tert- butoxy)-2-(4- (4,4-dimethylpiperidin- 1-yl)- 5-(4-((4-fluoro-N-(4- fluorobenzyl)benzamido) methyl)phenyl)-2,6- dimethylpyridin-3-yl) acetate | 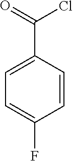 | 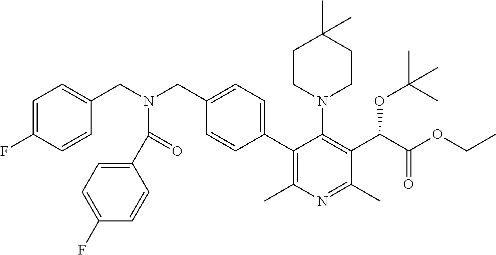 [SMILES: CCOC(=O)[C@@H](OC(C)(C)C)c1c(C)nc(C)c(-c2ccc(CN(Cc3ccc(F)cc3)C(=O)c3ccc(F)cc3)cc2)c1N1CCC(C)(C)CC1] | 712.5 |
| (S)-ethyl 2-(tert- butoxy)-2-(4- (4,4-dimethyl- piperidin-1-yl)- 5-(4-((N-(4-fluorobenzyl)- 2,5-dimethylfuran-3- carboxamido) methyl)phenyl)- 2,6-dimethylpyridin-3- yl)acetate | 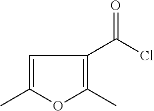 | 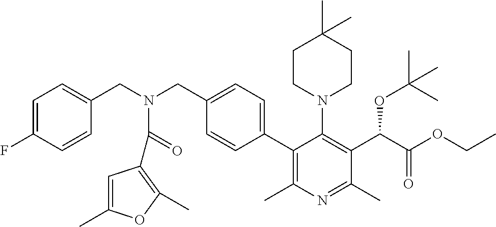 [SMILES: CCOC(=O)[C@@H](OC(C)(C)C)c1c(C)nc(C)c(-c2ccc(CN(Cc3ccc(F)cc3)C(=O)c3cc(C)oc3C)cc2)c1N1CCC(C)(C)CC1] | 712.5 |
| (S)-ethyl 2-(tert- butoxy)-2-(4- (4,4-dimethylpiperidin- 1-yl)-5-(4- ((N-(4-fluorobenzyl)-2- phenoxyacetamido)methyl) phenyl)-2,6-dimethyl- pyridin-3-yl)acetate | 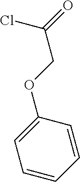 [SMILES: O=C(Cl)COc1ccccc1] | 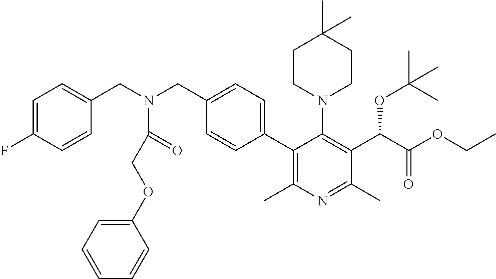 [SMILES: CCOC(=O)[C@@H](OC(C)(C)C)c1c(C)nc(C)c(-c2ccc(CN(Cc3ccc(F)cc3)C(=O)COc3ccccc3)cc2)c1N1CCC(C)(C)CC1] | 724.6 |
| (S)-ethyl 2-(5-(4- ((((benzyloxy)carbonyl)(4- fluorobenzyl)amino) methyl)phenyl)-4-(4,4- dimethylpiperidin-1-yl)-2,6- dimethylpyridin-3-yl)-2- (tert-butoxy)acetate | 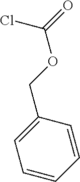 [SMILES: O=C(Cl)OCc1ccccc1] | 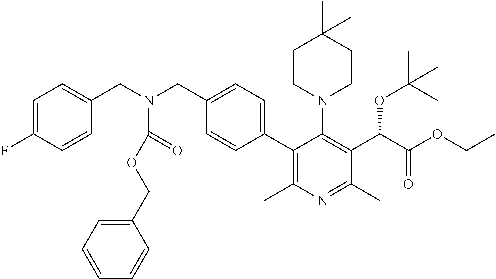 [SMILES: CCOC(=O)[C@@H](OC(C)(C)C)c1c(C)nc(C)c(-c2ccc(CN(Cc3ccc(F)cc3)C(=O)OCc3ccccc3)cc2)c1N1CCC(C)(C)CC1] | 724.5 |
| (S)-ethyl 2-(tert- butoxy)-2-(4- (4,4-dimethyl- piperidin-1-yl)-5-(4- (((ethoxycarbonyl)(4- fluorobenzyl)amino) methyl) phenyl)-2,6-dimethyl- pyridin-3-yl)acetate | 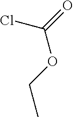 | 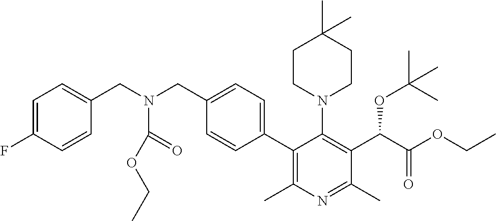 [SMILES: CCOC(=O)[C@@H](OC(C)(C)C)c1c(C)nc(C)c(-c2ccc(CN(Cc3ccc(F)cc3)C(=O)OCC)cc2)c1N1CCC(C)(C)CC1] | 662.5 |
| (S)-ethyl 2-(tert- butoxy)-2-(4- (4,4-dimethylpiperidin- 1-yl)- 5-(4-((N-(4-fluorobenzyl) methylsulfonamido) methyl)phenyl)-2,6- dimethylpyridin-3- yl)acetate | 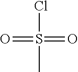 | 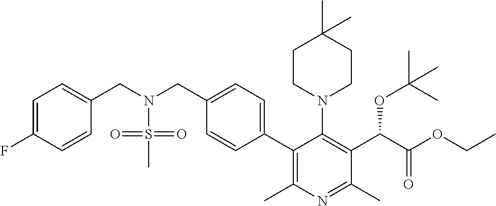 [SMILES: CCOC(=O)[C@@H](OC(C)(C)C)c1c(C)nc(C)c(-c2ccc(CN(Cc3ccc(F)cc3)S(C)(=O)=O)cc2)c1N1CCC(C)(C)CC1] | 668.4 |
| (S)-ethyl 2-(tert- butoxy)-2-(4- (4,4-dimethylpiperidin- 1-yl)-5-(4-((N-(4- fluorobenzyl)pyrrolidine-1- carboxamido)methyl) phenyl)-2,6-dimethyl- pyridin-3-yl)acetate | 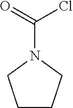 | 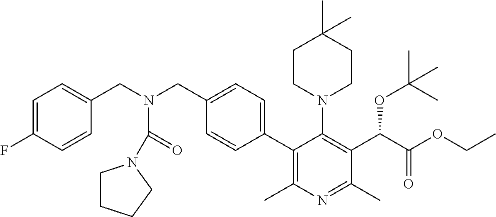 [SMILES: CCOC(=O)[C@@H](OC(C)(C)C)c1c(C)nc(C)c(-c2ccc(CN(Cc3ccc(F)cc3)C(=O)N3CCCC3)cc2)c1N1CCC(C)(C)CC1] | 687.5 |
| (S)-ethyl 2-(tert- butoxy)-2-(4- (4,4-dimethylpiperidin- 1-yl)-5-(4-((N- (4-fluorobenzyl)-3- methoxybenzamido) methyl)phenyl)-2,6- dimethylpyridin- 3-yl)acetate | 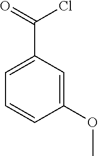 | 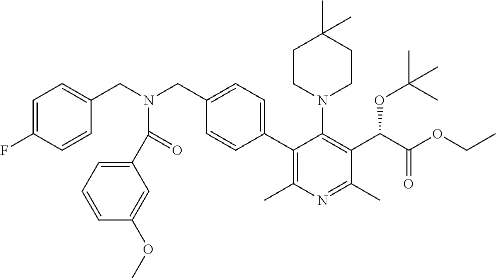 [SMILES: CCOC(=O)[C@@H](OC(C)(C)C)c1c(C)nc(C)c(-c2ccc(CN(Cc3ccc(F)cc3)C(=O)c3cccc(OC)c3)cc2)c1N1CCC(C)(C)CC1] | 724.5 |
| (S)-ethyl 2-(tert- butoxy)-2-(4- (4,4-dimethyl- piperidin-1-yl)-5-(4- ((N-(4-fluorobenzyl)-3- (trifluoromethyl) benzamido)methyl) phenyl)-2,6- dimethylpyridin-3-yl) acetate | 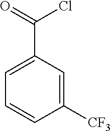 [SMILES: O=C(Cl)c1cccc(C(F)(F)F)c1] | 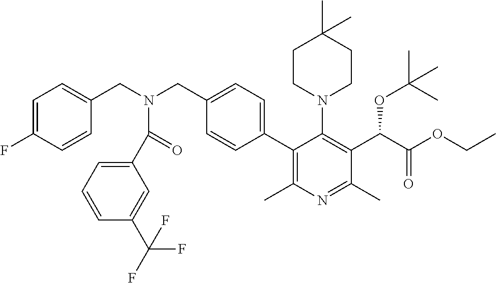 [SMILES: CCOC(=O)[C@@H](OC(C)(C)C)c1c(C)nc(C)c(-c2ccc(CN(Cc3ccc(F)cc3)C(=O)c3cccc(C(F)(F)F)c3)cc2)c1N1CCC(C)(C)CC1] | 762.5 |
| (S)-ethyl 2-(tert- butoxy)-2-(4- (4,4-dimethylpiperidin- 1-yl)-5-(4- ((N-(4-fluorobenzyl)-2- (trifluoromethyl) benzamido)methyl) phenyl)-2,6-dimethyl- pyridin-3-yl)acetate | 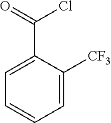 [SMILES: O=C(Cl)c1ccccc1C(F)(F)F] | 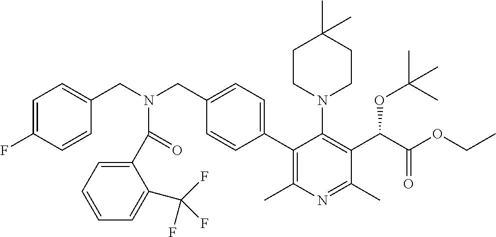 [SMILES: CCOC(=O)[C@@H](OC(C)(C)C)c1c(C)nc(C)c(-c2ccc(CN(Cc3ccc(F)cc3)C(=O)c3ccccc3C(F)(F)F)cc2)c1N1CCC(C)(C)CC1] | 762.5 |
| (S)-ethyl 2-(tert- butoxy)-2-(4- (4,4-dimethylpiperidin- 1-yl)-5-(4-((N-(4- fluorobenzyl) cyclopropane- carboxamido)methyl) phenyl)-2,6-dimethyl- pyridin-3-yl)acetate | 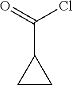 | 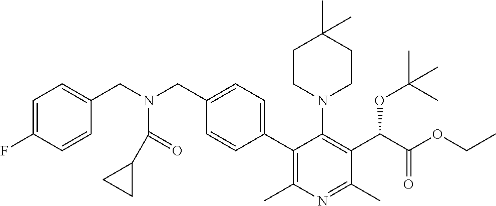 [SMILES: CCOC(=O)[C@@H](OC(C)(C)C)c1c(C)nc(C)c(-c2ccc(CN(Cc3ccc(F)cc3)C(=O)C3CC3)cc2)c1N1CCC(C)(C)CC1] | 658.6 |
| (S)-ethyl 2-(tert- butoxy)-2-(4- (4,4-dimethylpiperidin- 1-yl)-5-(4-((3-fluoro-N-(4- fluorobenzyl)benzamido) methyl)phenyl)-2,6- dimethylpyridin-3-yl) acetate | 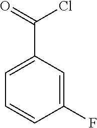 | 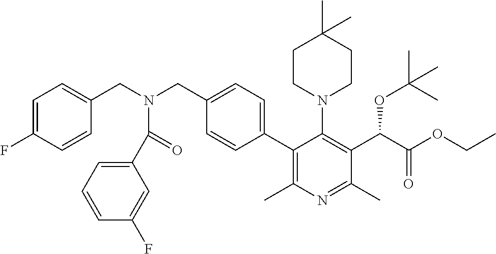 [SMILES: CCOC(=O)[C@@H](OC(C)(C)C)c1c(C)nc(C)c(-c2ccc(CN(Cc3ccc(F)cc3)C(=O)c3cccc(F)c3)cc2)c1N1CCC(C)(C)CC1] | 712.6 |
| (S)-ethyl 2-(tert- butoxy)-2-(4- (4,4-dimethylpiperidin- 1-yl)- 5-(4-((N-(4-fluorobenzyl) cyclobutanecarbox- amido)methyl)phenyl)-2,6- dimethylpyridin-3-yl) acetate | 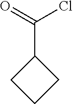 | 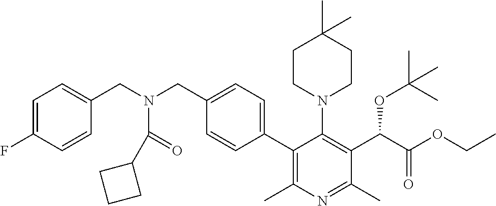 [SMILES: CCOC(=O)[C@@H](OC(C)(C)C)c1c(C)nc(C)c(-c2ccc(CN(Cc3ccc(F)cc3)C(=O)C3CCC3)cc2)c1N1CCC(C)(C)CC1] | 672.6 |
| (S)-ethyl 2-(tert- butoxy)-2-(4-(4,4- dimethylpiperidin-1-yl)- 5-(4-((N-(4- fluorobenzyl)-2- methylbenzamido) methyl)phenyl)- 2,6-dimethylpyridin-3- yl)acetate | 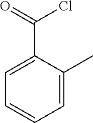 | 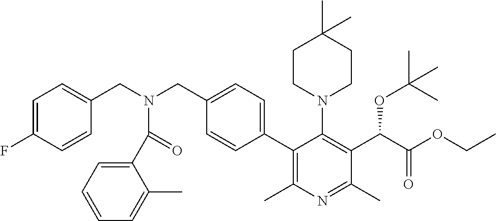 [SMILES: CCOC(=O)[C@@H](OC(C)(C)C)c1c(C)nc(C)c(-c2ccc(CN(Cc3ccc(F)cc3)C(=O)c3ccccc3C)cc2)c1N1CCC(C)(C)CC1] | 708.5 |
| (S)-ethyl 2-(tert- butoxy)-2-(4- (4,4-dimethylpiperidin- 1-yl)-5-(4-((N-(4- fluorobenzyl)thiophene-2- carboxamido)methyl) phenyl)-2,6-dimethyl- pyridin-3-yl)acetate | 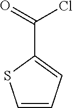 | 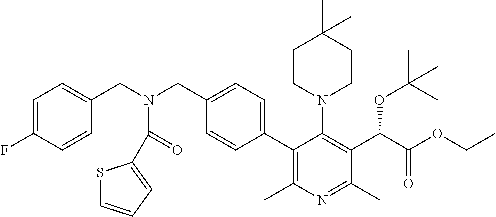 [SMILES: CCOC(=O)[C@@H](OC(C)(C)C)c1c(C)nc(C)c(-c2ccc(CN(Cc3ccc(F)cc3)C(=O)c3cccs3)cc2)c1N1CCC(C)(C)CC1] | 700.5 |
| (S)-ethyl 2-(tert- butoxy)-2-(4- (4,4-dimethylpiperidin- 1-yl)-5-(4- ((N-(4-fluorobenzyl)-4- methoxybenzamido) methyl) phenyl)-2,6-dimethyl- pyridin-3-yl)acetate | 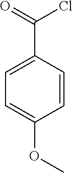 | 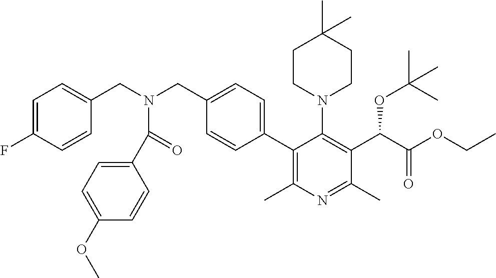 [SMILES: CCOC(=O)[C@@H](OC(C)(C)C)c1c(C)nc(C)c(-c2ccc(CN(Cc3ccc(F)cc3)C(=O)c3ccc(OC)cc3)cc2)c1N1CCC(C)(C)CC1] | 724.6 |
| (S)-ethyl 2-(tert- butoxy)-2-(4- (4,4-dimethylpiperidin- 1-yl)-5-(4- ((N-(4-fluorobenzyl)-4- (trifluoromethyl) benzamido) methyl)phenyl)-2,6- dimethylpyridin-3-yl) acetate | 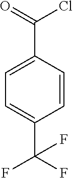 [SMILES: O=C(Cl)c1ccc(C(F)(F)F)cc1] | 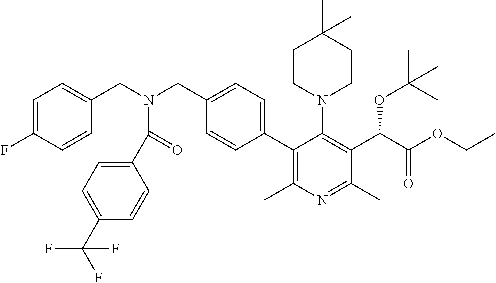 [SMILES: CCOC(=O)[C@@H](OC(C)(C)C)c1c(C)nc(C)c(-c2ccc(CN(Cc3ccc(F)cc3)C(=O)c3ccc(C(F)(F)F)cc3)cc2)c1N1CCC(C)(C)CC1] | 762.5 |
| (S)-ethyl 2-(tert- butoxy)-2-(4- (4,4-dimethylpiperidin- 1-yl)- 5-(4-((N-(4-fluorobenzyl)- 2,4,6-trimethyl- phenylsulfonamido) methyl)phenyl)-2,6- dimethylpyridin-3-yl) acetate | 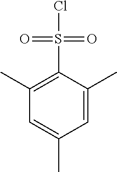 | 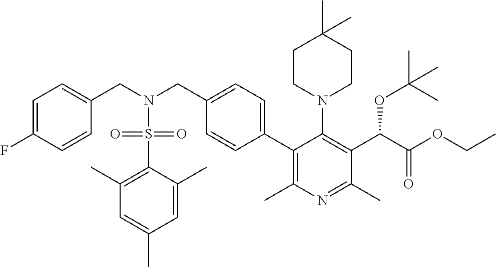 [SMILES: CCOC(=O)[C@@H](OC(C)(C)C)c1c(C)nc(C)c(-c2ccc(CN(Cc3ccc(F)cc3)S(=O)(=O)c3c(C)cc(C)cc3C)cc2)c1N1CCC(C)(C)CC1] | 772.5 |

Step 2: General procedure: NaOH (3 eq.) was added to a solution of the ester obtained in the step 1 (1 eq.) in EtOH or MeOH and water (volume ratio 1:1). The reaction was heated at 85° C. for 1-2 h. The desired acid was isolated by the preparative HPLC system.

| | | LCMS |
|---|---|---|
| Name | Structure | (M + H) |
| (S)-2-(tert-butoxy)-2-(4- (4,4-dimethylpiperidin- 1-yl)-5-(4-((N-(4- fluorobenzyl)cyclopentane- carboxamido)methyl) phenyl)-2,6- dimethylpyridin-3- yl)acetic acid | 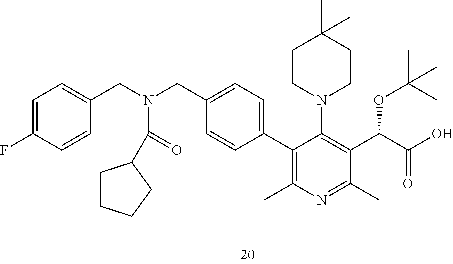 [SMILES: Cc1nc(C)c([C@H](OC(C)(C)C)C(=O)O)c(N2CCC(C)(C)CC2)c1-c1ccc(CN(Cc2ccc(F)cc2)C(=O)C2CCCC2)cc1] | 658.5 |
| (S)-2-(tert-butoxy)-2-(4- (4,4-dimethylpiperidin- 1-yl)-5-(4-((N-(4- fluorobenzyl)benamido) methyl)phenyl)-2,6- dimethylpyridin-3- yl)acetic acid | 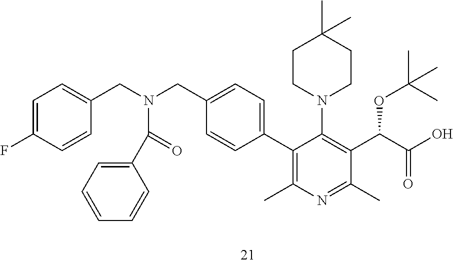 [SMILES: Cc1nc(C)c([C@H](OC(C)(C)C)C(=O)O)c(N2CCC(C)(C)CC2)c1-c1ccc(CN(Cc2ccc(F)cc2)C(=O)c2ccccc2)cc1] | 666.4 |
| (S)-2-(tert-butoxy)-2-(4- (4,4-dimethylpiperidin- 1-yl)-5-(4-((N-(4- fluorobenzyl)propionamido) methyl)phenyl)-2,6- dimethylpyridin-3- yl)acetic acid | 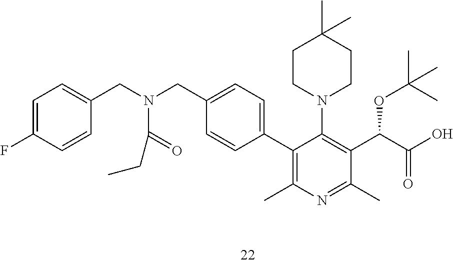 [SMILES: CCC(=O)N(Cc1ccc(F)cc1)Cc1ccc(-c2c(C)nc(C)c([C@H](OC(C)(C)C)C(=O)O)c2N2CCC(C)(C)CC2)cc1] | 618.4 |
| (S)-2-(tert-butoxy)-2-(4- (4,4-dimethylpiperidin- 1-yl)-5-(4-((N-(4- fluorobenzyl)isobutyramido) methyl)phenyl)-2,6- dimethylpyridin-3- yl)acetic acid | 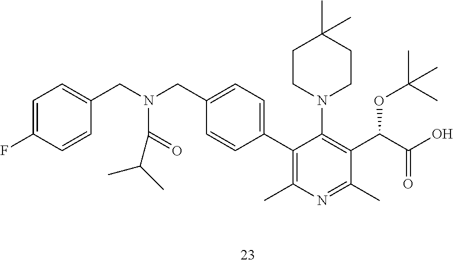 [SMILES: Cc1nc(C)c([C@H](OC(C)(C)C)C(=O)O)c(N2CCC(C)(C)CC2)c1-c1ccc(CN(Cc2ccc(F)cc2)C(=O)C(C)C)cc1] | 632.4 |
| (S)-2-(tert-butoxy)-2-(4- (4,4-dimethylpiperidin- 1-yl)-5-(4-((N-(4- fluorobenzyl)acetamido) methyl)phenyl)-2,6- dimethylpyridin-3- yl)acetic acid | 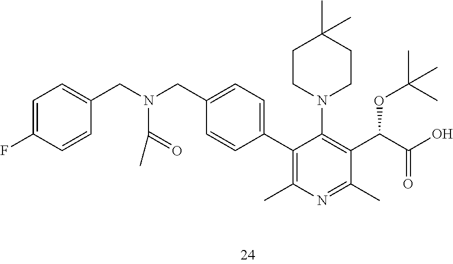 [SMILES: CC(=O)N(Cc1ccc(F)cc1)Cc1ccc(-c2c(C)nc(C)c([C@H](OC(C)(C)C)C(=O)O)c2N2CCC(C)(C)CC2)cc1] | 604.4 |
| (S)-2-(tert-butoxy)-2-(4- (4,4-dimethylpiperidin- 1-yl)-5-(4-((N-(4- fluorobenzyl)pivalamido) methyl)phenyl)-2,6- dimethylpyridin-3- yl)acetic acid | 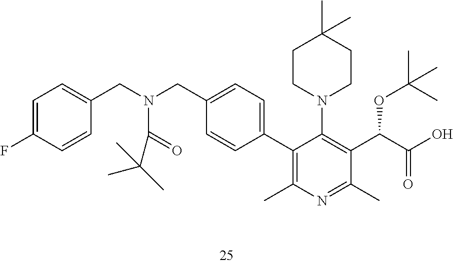 [SMILES: Cc1nc(C)c([C@H](OC(C)(C)C)C(=O)O)c(N2CCC(C)(C)CC2)c1-c1ccc(CN(Cc2ccc(F)cc2)C(=O)C(C)(C)C)cc1] | 646.4 |
| (S)-2-(tert-butoxy)-2-(4- (4,4-dimethylpiperidin- 1-yl)-5-(4-((N-(4- fluorobenzyl)-2- methoxybenzamido)meth- yl)phenyl)-2,6- dimethylpyridin-3- yl)acetic acid | 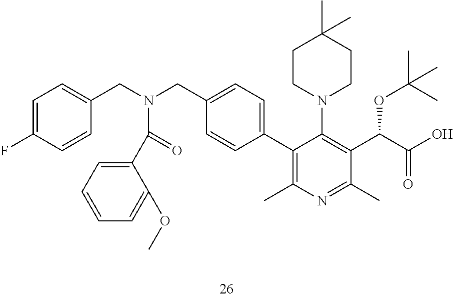 [SMILES: COc1ccccc1C(=O)N(Cc1ccc(F)cc1)Cc1ccc(-c2c(C)nc(C)c([C@H](OC(C)(C)C)C(=O)O)c2N2CCC(C)(C)CC2)cc1] | 696.5 |
| (S)-2-(tert-butoxy)-2-(4- (4,4-dimethylpiperidin- 1-yl)-5-(4-((2-fluoro-N- (4- fluorobenzyl)benzamido) methyl)phenyl)-2,6- dimethylpyridin-3- yl)acetic acid | 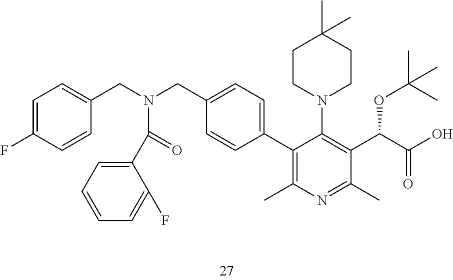 [SMILES: Cc1nc(C)c([C@H](OC(C)(C)C)C(=O)O)c(N2CCC(C)(C)CC2)c1-c1ccc(CN(Cc2ccc(F)cc2)C(=O)c2ccccc2F)cc1] | 684.4 |
| (S)-2-(tert-butoxy)-2-(4- (4,4-dimethylpiperidin- 1-yl)-5-(4-((4-fluoro-N- (4- fluorobenzyl)benzamido) methyl)phenyl)-2,6- dimethylpyridin-3- yl)acetic acid | 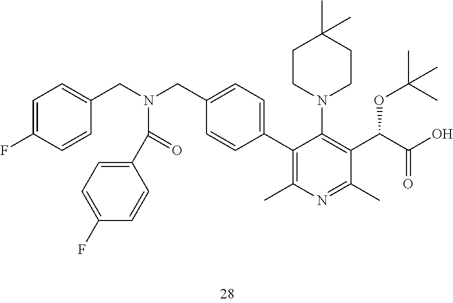 [SMILES: Cc1nc(C)c([C@H](OC(C)(C)C)C(=O)O)c(N2CCC(C)(C)CC2)c1-c1ccc(CN(Cc2ccc(F)cc2)C(=O)c2ccc(F)cc2)cc1] | 684.4 |
| (S)-2-(terr-butoxy)-2-(4- (4,4-dimethylpiperidin- 1-yl)-5-(4-((N-(4- fluorobenzyl)-2,5- dimethylfuran-3- carboxamido)methyl) phenyl)-2,6- dimethylpyridin-3- yl)acetic acid | 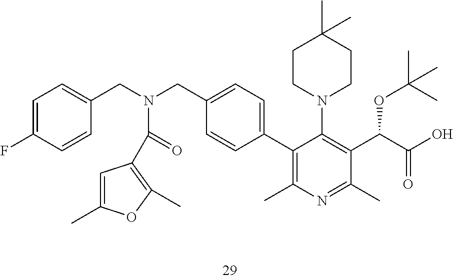 [SMILES: Cc1cc(C(=O)N(Cc2ccc(F)cc2)Cc2ccc(-c3c(C)nc(C)c([C@H](OC(C)(C)C)C(=O)O)c3N3CCC(C)(C)CC3)cc2)c(C)o1] | 684.4 |
| (S)-2-(tert-butoxy)-2-(4- (4,4-dimethylpiperidin- 1-yl)-5-(4-((N-(4- fluorobenzyl)-2- phenoxyacetamido)meth- yl)phenyl)-2,6- dimethylpyridin-3- yl)acetic acid | 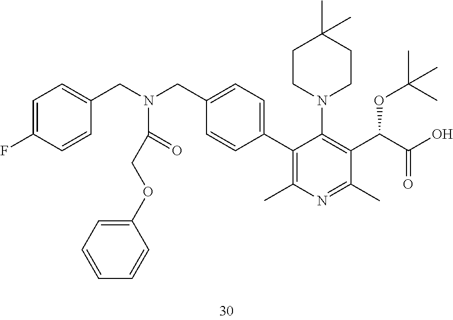 [SMILES: Cc1nc(C)c([C@H](OC(C)(C)C)C(=O)O)c(N2CCC(C)(C)CC2)c1-c1ccc(CN(Cc2ccc(F)cc2)C(=O)COc2ccccc2)cc1] | 696.4 |
| (S)-2-(5-(4- ((((benzyloxy)carbonyl) (4- fluorobenzyl)amino)meth- yl)phenyl)-4-(4,4- dimethylpiperidin-1-yl)- 2,6-dimethylpyridin-3- yl)-2-(tert-butoxy)acetic acid | 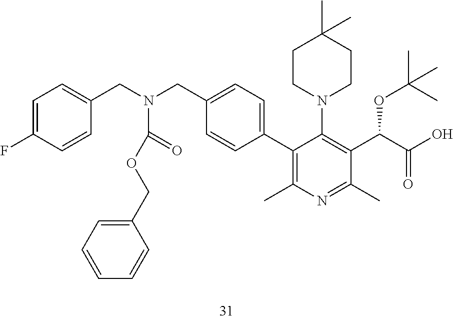 [SMILES: Cc1nc(C)c([C@H](OC(C)(C)C)C(=O)O)c(N2CCC(C)(C)CC2)c1-c1ccc(CN(Cc2ccc(F)cc2)C(=O)OCc2ccccc2)cc1] | 696.4 |
| (S)-2-(tert-butoxy)-2-(4- (4,4-dimethylpiperidin- 1-yl)-5-(4-(((4- fluorobenzyl)(methoxy- carbonyl)amino)methyl) phenyl)-2,6- dimethylpyridin-3- yl)acetic acid | 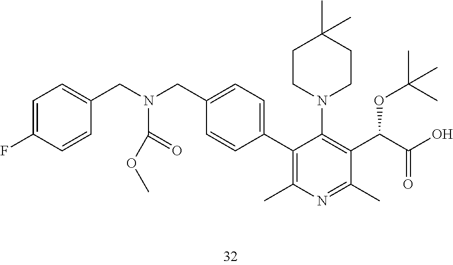 [SMILES: COC(=O)N(Cc1ccc(F)cc1)Cc1ccc(-c2c(C)nc(C)c([C@H](OC(C)(C)C)C(=O)O)c2N2CCC(C)(C)CC2)cc1] | 620.4 |
| (S)-2-(tert-butoxy)-2-(4- (4,4-dimethylpiperidin- 1-yl)-5-(4- (((ethoxycarbonyl)(4 fluorobenzyl)amino)meth- yl)phenyl)-2,6- dimethylpyridin-3- yl)acetic acid | 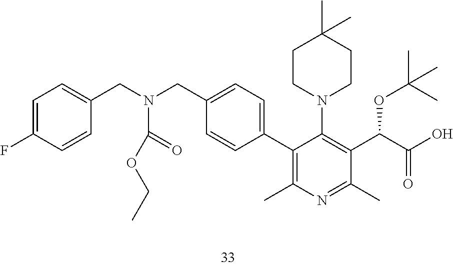 [SMILES: CCOC(=O)N(Cc1ccc(F)cc1)Cc1ccc(-c2c(C)nc(C)c([C@H](OC(C)(C)C)C(=O)O)c2N2CCC(C)(C)CC2)cc1] | 634.5 |
| (S)-2-(tert-butoxy)-2-(4- (4,4-dimethylpiperidin- 1-yl)-5-(4-((N-(4- fluorobenzyl)methyl- sulfonamido)methyl)phenyl)- 2,6-dimethylpyridin-3- yl)acetic acid | 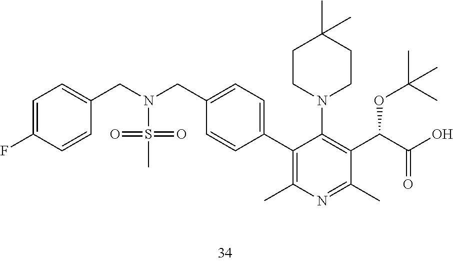 [SMILES: Cc1nc(C)c([C@H](OC(C)(C)C)C(=O)O)c(N2CCC(C)(C)CC2)c1-c1ccc(CN(Cc2ccc(F)cc2)S(C)(=O)=O)cc1] | 640.4 |
| (S)-2-(tert-butoxy)-2-(4- (4,4-dimethylpiperidin- 1-yl)-5-(4-((N-(4- fluorobenzyl)pyrrolidine- 1- carboxamido)methyl) phenyl)-2,6- dimethylpyridin-3- yl)acetic acid | 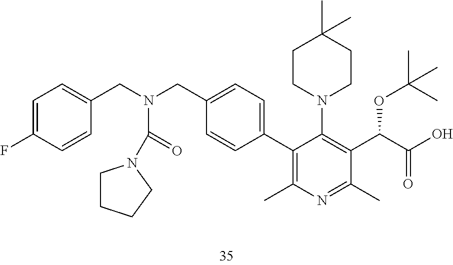 [SMILES: Cc1nc(C)c([C@H](OC(C)(C)C)C(=O)O)c(N2CCC(C)(C)CC2)c1-c1ccc(CN(Cc2ccc(F)cc2)C(=O)N2CCCC2)cc1] | 659.5 |
| (S)-2-(tert-butoxy)-2-(4- (4,4-dimethylpiperidin- 1-yl)-5-(4-((N-(4- fluorobenzyl)phenyl- sulfonamido)methyl)phenyl)- 2,6-dimethylpyridin-3- yl)acetic acid | 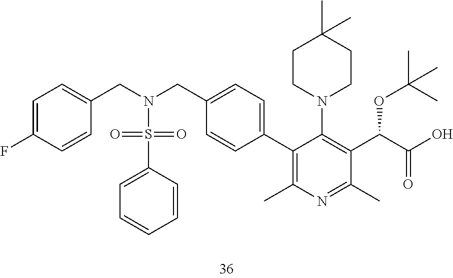 [SMILES: Cc1nc(C)c([C@H](OC(C)(C)C)C(=O)O)c(N2CCC(C)(C)CC2)c1-c1ccc(CN(Cc2ccc(F)cc2)S(=O)(=O)c2ccccc2)cc1] | 702.4 |
| (S)-2-(tert-butoxy)-2-(4- (4,4-dimethylpiperidin- 1-yl)-5-(4-((N-(4- fluorobenzyl)-3- methoxybenzamido)meth- yl)phenyl)-2,6- dimethylpyridin-3- yl)acetic acid | 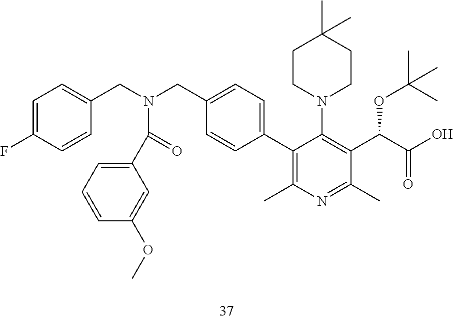 [SMILES: COc1cccc(C(=O)N(Cc2ccc(F)cc2)Cc2ccc(-c3c(C)nc(C)c([C@H](OC(C)(C)C)C(=O)O)c3N3CCC(C)(C)CC3)cc2)c1] | 696.5 |
| (S)-2-(tert-butoxy)-2-(4- (4,4-dimethylpiperidin- 1-yl)-5-(4-((N-(4- fluorobenzyl)-3- (trifluoromethyl)benz- amio)methyl)phenyl)-2,6- dimethylpyridin-3- yl)acetic acid | 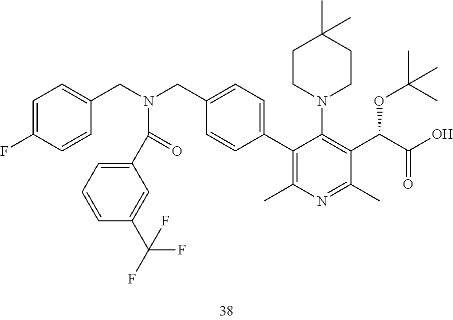 [SMILES: Cc1nc(C)c([C@H](OC(C)(C)C)C(=O)O)c(N2CCC(C)(C)CC2)c1-c1ccc(CN(Cc2ccc(F)cc2)C(=O)c2cccc(C(F)(F)F)c2)cc1] | 734.6 |
| (S)-2-(tert-butoxy)-2-(4- (4,4-dimethylpiperidin- 1-yl)-5-(4-((N-(4- fluorobenzyl)-2- (trifluoromethyl)benz- amido)methyl)phenyl)-2,6- dimethylpyridin-3- yl)acetic acid | 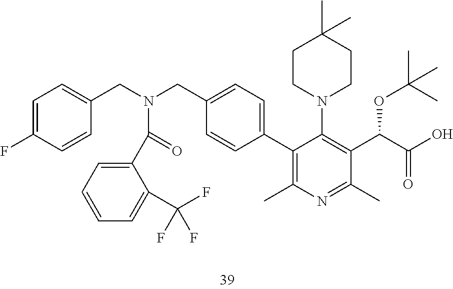 [SMILES: Cc1nc(C)c([C@H](OC(C)(C)C)C(=O)O)c(N2CCC(C)(C)CC2)c1-c1ccc(CN(Cc2ccc(F)cc2)C(=O)c2ccccc2C(F)(F)F)cc1] | 734.5 |
| (S)-2-(tert-butoxy)-2-(4- (4,4-dimethylpiperidin- 1-yl)-5-(4-((N-(4- fluorobenzyl)cyclopropane- carboxamido)methyl) phenyl)-2,6- dimethylpyridin-3- yl)acetic acid | 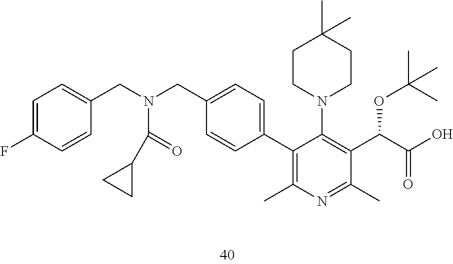 [SMILES: Cc1nc(C)c([C@H](OC(C)(C)C)C(=O)O)c(N2CCC(C)(C)CC2)c1-c1ccc(CN(Cc2ccc(F)cc2)C(=O)C2CC2)cc1] | 630.4 |
| (S)-2-(tert-butoxy)-2-(4- (4,4-dimethylpiperidin- 1-yl)-5-(4-((3-fluoro-N- (4- fluorobenzyl)benzamido) methyl)phenyl)-2,6- dimethylpyridin-3- yl)acetic acid | 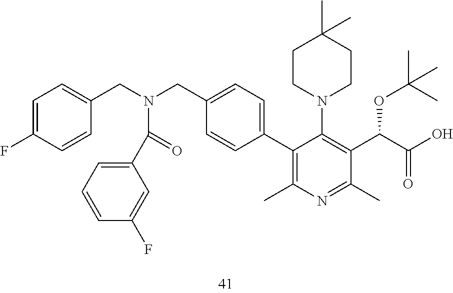 [SMILES: Cc1nc(C)c([C@H](OC(C)(C)C)C(=O)O)c(N2CCC(C)(C)CC2)c1-c1ccc(CN(Cc2ccc(F)cc2)C(=O)c2cccc(F)c2)cc1] | 684.4 |
| (S)-2-(tert-butoxy)-2-(4- (4,4-dimethylpiperidin- 1-yl)-5-(4-((N-(4- fluorobenzyl)cyclobutane- carboxamido)methyl) phenyl)-2,6- dimethylpyridin-3- yl)acetic acid | 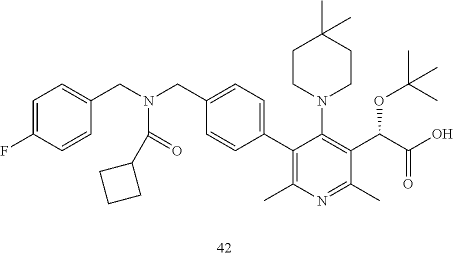 [SMILES: Cc1nc(C)c([C@H](OC(C)(C)C)C(=O)O)c(N2CCC(C)(C)CC2)c1-c1ccc(CN(Cc2ccc(F)cc2)C(=O)C2CCC2)cc1] | 644.4 |
| (S)-2-(tert-butoxy)-2-(4- (4,4-dimethylpiperidin- 1-yl)-5-(4-((N-(4- fluorobenzyl)-2- methylbenzamido)methyl) phenyl)-2,6- dimethylpyridin-3- yl)acetic acid | 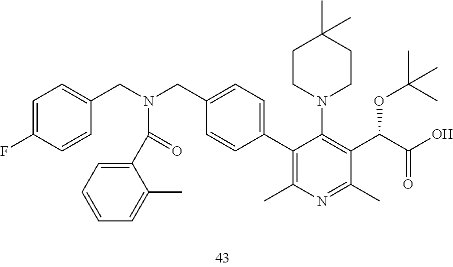 [SMILES: Cc1ccccc1C(=O)N(Cc1ccc(F)cc1)Cc1ccc(-c2c(C)nc(C)c([C@H](OC(C)(C)C)C(=O)O)c2N2CCC(C)(C)CC2)cc1] | 680.6 |
| (S)-2-(tert-butoxy)-2-(4- (4,4-dimethylpiperidin- 1-yl)-5-(4-((N-(4- fluorobenzyl)thiophene- 2- carboxamido)methyl) phenyl)-2,6- dimethylpyridin-3- yl)acetic acid | 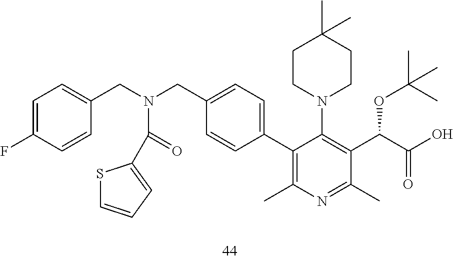 [SMILES: Cc1nc(C)c([C@H](OC(C)(C)C)C(=O)O)c(N2CCC(C)(C)CC2)c1-c1ccc(CN(Cc2ccc(F)cc2)C(=O)c2cccs2)cc1] | 672.5 |
| (S)-2-(tert-butoxy)-2-(4- (4,4-dimethylpiperidin- 1-yl)-5-(4-((N-(4- fluorobenzyl)-4- methoxybenzamido)meth- yl)phenyl)-2,6- dimethylpyridin-3- yl)acetic acid | 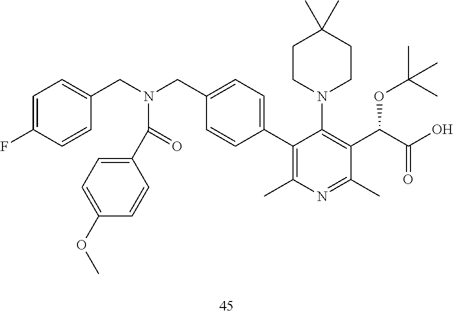 [SMILES: COc1ccc(C(=O)N(Cc2ccc(F)cc2)Cc2ccc(-c3c(C)nc(C)c([C@H](OC(C)(C)C)C(=O)O)c3N3CCC(C)(C)CC3)cc2)cc1] | 696.6 |
| (S)-2-(tert-butoxy)-2-(4- (4,4-dimethylpiperidin- 1-yl)-5-(4-((N-(4- fluorobenzyl)-4- (trifluoromethyl)benz- amido)methyl)phenyl)- 2,6-dimethylpyridin-3- yl)acetic acid | 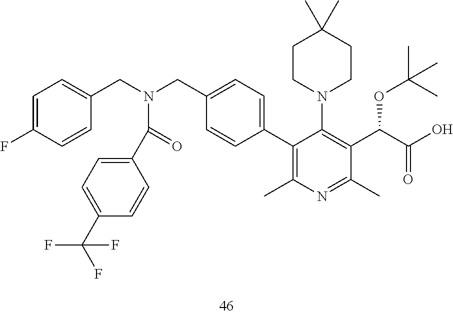 [SMILES: Cc1nc(C)c([C@H](OC(C)(C)C)C(=O)O)c(N2CCC(C)(C)CC2)c1-c1ccc(CN(Cc2ccc(F)cc2)C(=O)c2ccc(C(F)(F)F)cc2)cc1] | 734.4 |
| (S)-2-(tert-butoxy)-2-(4- (4,4-dimethylpiperidin- 1-yl)-5-(4-((N-(4- fluorobenzyl)-2,4,6- trimethylphenylsulfon- amido)methyl)phenyl)-2,6- dimethylpyridin-3- yl)acetic acid | 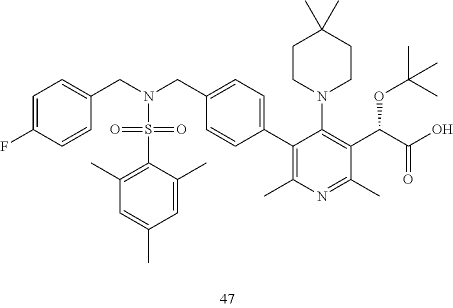 [SMILES: Cc1cc(C)c(S(=O)(=O)N(Cc2ccc(F)cc2)Cc2ccc(-c3c(C)nc(C)c([C@H](OC(C)(C)C)C(=O)O)c3N3CCC(C)(C)CC3)cc2)c(C)c1] | 744.5 |

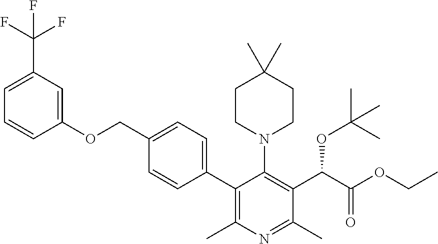 [SMILES: CCOC(=O)[C@@H](OC(C)(C)C)c1c(C)nc(C)c(-c2ccc(COc3cccc(C(F)(F)F)c3)cc2)c1N1CCC(C)(C)CC1]

### (S)-Ethyl 2-(tert-butoxy)-2-(4-(4,4-dimethylpiperidin-1-yl)-2,6-dimethyl-5-(4-((3-(trifluoromethyl)phenoxy)methyl)phenyl)pyridin-3-yl)acetate

A mixture of (S)-ethyl 2-(5-bromo-4-(4,4-dimethylpiperidin-1-yl)-2,6-dimethylpyridin-3-yl)-2-(tert-butoxy)acetate (0.0313 g, 0.069 mmol), (4-((3-(trifluoromethyl)phenoxy)methyl)phenyl)boronic acid (0.031 g, 0.103 mmol) and 2M Na2CO3 (0.086 ml, 0.172 mmol) in DMF (2 mL) was degassed for 10 min. Then, Pd(Ph3P)4 (7.94 mg, 6.87 μmol) was added, degassed for 5 min and placed in a pre-heated oil bath at 110° C. After 2 h, cooled and purified by prep-HPLC to afford (S)-ethyl 2-(tert-butoxy)-2-(4-(4,4-dimethylpiperidin-1-yl)-2,6-dimethyl-5-(4-((3-(trifluoromethyl)phenoxy)methyl)phenyl)pyridin-3-yl)acetate (0.025 g, 0.040 mmol, 58.0% yield) as white solid. ¹H NMR (500 MHz, CDCl₃) δ 7.50-7.55 (m, 2H), 7.40-7.45 (m, 1H), 7.33 (dd, J=1.5, 7.8 Hz, 1H), 7.24-7.28 (m, 2H), 7.22 (dd, J=1.5, 7.8 Hz, 1H), 7.16-7.20 (m, 1H), 6.07 (s, 1H), 5.22 (s, 2H), 4.27 (qd, J=7.1, 10.7 Hz, 1H), 4.18 (qd, J=7.1, 10.7 Hz, 1H), 3.21 (d, J=11.2 Hz, 1H), 2.86 (t, J=12.0 Hz, 1H), 2.62 (s, 3H), 2.24-2.31 (m, 1H), 2.21 (s, 3H), 1.97 (t, J=11.5 Hz, 1H), 1.50-1.57 (m, 1H), 1.32-1.39 (m, 1H), 1.27 (t, J=7.1 Hz, 3H), 1.21 (s, 9H), 1.14-1.20 (m, 1H), 1.04 (d, J=12.8 Hz, 1H), 0.89 (s, 3H), 0.56 (s, 3H). LCMS (M+H)=627.4.

### Example 48

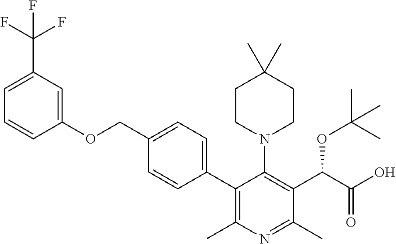 [SMILES: Cc1nc(C)c([C@H](OC(C)(C)C)C(=O)O)c(N2CCC(C)(C)CC2)c1-c1ccc(COc2cccc(C(F)(F)F)c2)cc1]

### (S)-2-(tert-Butoxy)-2-(4-(4,4-dimethylpiperidin-1-yl)-2,6-dimethyl-5-(4-((3-(trifluoromethyl)phenoxy)methyl)phenyl)pyridin-3-yl)acetic acid

A mixture of (S)-ethyl 2-(tert-butoxy)-2-(4-(4,4-dimethylpiperidin-1-yl)-2,6-dimethyl-5-(4-((3-(trifluoromethyl)phenoxy)methyl)phenyl)pyridin-3-yl)acetate (0.023 g, 0.037 mmol) and LiOH (8.79 mg, 0.367 mmol) in 9:1 EtOH/H2O (2 mL) was refluxed for 3 h. Then, cooled and purified to afford (S)-2-(tert-butoxy)-2-(4-(4,4-dimethylpiperidin-1-yl)-2,6-dimethyl-5-(4-((3-(trifluoromethyl)phenoxy)methyl)phenyl)pyridin-3-yl)acetic acid (0.0196 g, 0.033 mmol, 89% yield) as solid. ¹HNMR (500 MHz, CDCl₃) δ 7.53 (t, J=5.8 Hz, 2H), 7.39-7.45 (m, 1H), 7.29-7.33 (m, 1H), 7.24-7.28 (m, 2H), 7.15-7.21 (m, 2H), 5.82 (br. s., 1H), 5.21 (s, 2H), 2.73 (s, 3H), 2.25 (s, 3H), 1.25-1.41 (m, 4H), 1.23 (s, 9H), 0.84 (m, 6H). 4H of piperidine were not resolved. LCMS (M+H)=599.47.

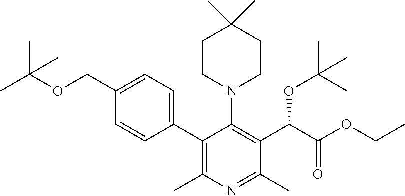 [SMILES: CCOC(=O)[C@@H](OC(C)(C)C)c1c(C)nc(C)c(-c2ccc(COC(C)(C)C)cc2)c1N1CCC(C)(C)CC1]

### (S)-Ethyl 2-(tert-butoxy)-2-(5-(4-(tert-butoxymethyl)phenyl)-4-(4,4-dimethylpiperidin-1-yl)-2,6-dimethylpyridin-3-yl)acetate

A mixture of (S)-ethyl 2-(5-bromo-4-(4,4-dimethylpiperidin-1-yl)-2,6-dimethylpyridin-3-yl)-2-(tert-butoxy)acetate (0.0475 g, 0.104 mmol), (4-(tert-butoxymethyl)phenyl)boronic acid (0.033 g, 0.156 mmol) and 2M Na2CO3 (0.130 ml, 0.261 mmol) in DMF (2 mL) was degassed for 10 min. Then, Pd(Ph3P)4 (0.012 g, 10.43 μmol) was added, degassed for 5 min and placed in a pre-heated oil bath at 110° C. After 2 h, cooled and purified by prep-HPLC to afford (S)-ethyl 2-(tert-butoxy)-2-(5-(4-(tert-butoxymethyl)phenyl)-4-(4,4-dimethylpiperidin-1-yl)-2,6-dimethylpyridin-3-yl)acetate (0.021 g, 0.039 mmol, 37.4% yield) as white solid. ¹H NMR (500 MHz, CDCl₃) δ 7.40-7.45 (m, 2H), 7.23 (dd, J=1.6, 7.9 Hz, 1H), 7.11-7.15 (dd, J=1.6, 7.9 Hz, 1H), 6.08 (s, 1H), 4.56 (s, 2H), 4.26 (qd, J=7.1, 10.7 Hz, 1H), 4.18 (qd, J=7.1, 10.7 Hz, 1H), 3.20 (d, J=12.0 Hz, 1H), 2.88 (t, J=12.0 Hz, 1H), 2.61 (s, 3H), 2.26 (d, J=11.8 Hz, 1H), 2.19 (s, 3H), 2.00 (t, J=11.6 Hz, 1H), 1.55 (dt, J=4.0, 12.5 Hz, 1H), 1.32-1.39 (m, 1H), 1.34 (s, 9H), 1.26 (t, J=7.1 Hz, 3H), 1.21 (s, 9H), 1.16-1.20 (m, 1H), 1.05 (d, J=12.5 Hz, 1H), 0.89 (s, 3H), 0.62 (s, 3H). LCMS (M+H)=539.5.

### Example 49

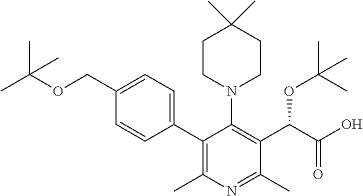 [SMILES: Cc1nc(C)c([C@H](OC(C)(C)C)C(=O)O)c(N2CCC(C)(C)CC2)c1-c1ccc(COC(C)(C)C)cc1]

### (S)-2-(tert-Butoxy)-2-(5-(4-(tert-butoxymethyl)phenyl)-4-(4,4-dimethylpiperidin-1-yl)-2,6-dimethylpyridin-3-yl)acetic acid

A mixture of (S)-ethyl 2-(tert-butoxy)-2-(5-(4-(tert-butoxymethyl)phenyl)-4-(4,4-dimethylpiperidin-1-yl)-2,6-dimethylpyridin-3-yl)acetate (0.021 g, 0.039 mmol) and LiOH (9.33 mg, 0.390 mmol) in 9:1 EtOH/H2O (2 mL) was refluxed for 3 h. Then, cooled and purified by prep-HPLC to afford (S)-2-(tert-butoxy)-2-(5-(4-(tert-butoxymethyl)phenyl)-4-(4,4-dimethylpiperidin-1-yl)-2,6-dimethylpyridin-3-yl)acetic acid (0.0172 g, 0.034 mmol, 86% yield) as light brown solid. ¹H NMR (400 MHz, CDCl₃) δ 7.42 (t, J=7.2 Hz, 2H), 7.20 (d, J=7.8 Hz, 1H), 7.04 (d, J=7.8 Hz, 1H), 5.65 (br. s., 1H), 4.54 (s, 2H), 2.81 (s, 3H), 2.26 (s, 3H), 1.32 (s, 9H), 1.22-1.30 (m, 4H), 1.20 (s, 9H), 0.75 (br. s., 6H). 4H of piperidine were not resolved. LCMS (M+H)=511.4.

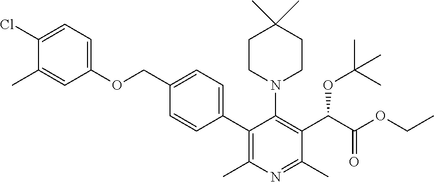 [SMILES: CCOC(=O)[C@@H](OC(C)(C)C)c1c(C)nc(C)c(-c2ccc(COc3ccc(Cl)c(C)c3)cc2)c1N1CCC(C)(C)CC1]

### (S)-Ethyl 2-(tert-butoxy)-2-(5-(4-((4-chloro-3-methylphenoxy)methyl)phenyl)-4-(4,4-dimethylpiperidin-1-yl)-2,6-dimethylpyridin-3-yl)acetate

A mixture of (S)-ethyl 2-(5-bromo-4-(4,4-dimethylpiperidin-1-yl)-2,6-dimethylpyridin-3-yl)-2-(tert-butoxy)acetate (0.0423 g, 0.093 mmol), (4-((4-chloro-3-methylphenoxy)methyl)phenyl)boronic acid (0.039 g, 0.139 mmol) and 2M Na2CO3 (0.116 ml, 0.232 mmol) in DMF (2 mL) was degassed for 10 min. Then, Pd(Ph3P)4 (10.73 mg, 9.29 μmol) was added, degassed for 5 min and placed in a pre-heated oil bath at 110° C. After 2 h, cooled and purified by pre-HPLC to afford (S)-ethyl 2-(tert-butoxy)-2-(5-(4-((4-chloro-3-methylphenoxy)methyl)phenyl)-4-(4,4-dimethylpiperidin-1-yl)-2,6-dimethylpyridin-3-yl)acetate (0.033 g, 0.054 mmol, 58.5% yield) as white solid. ¹H NMR (500 MHz, CDCl₃) δ 7.47-7.52 (m, 2H), 7.29-7.32 (m, 1H), 7.24 (d, J=8.8 Hz, 1H), 7.18-7.22 (m, 1H), 6.91 (d, J=2.5 Hz, 1H), 6.76-6.79 (m, 1H), 6.07 (s, 1H), 5.14 (s, 2H), 4.23-4.31 (m, 1H), 4.18 (qd, J=7.1, 10.9 Hz, 1H), 3.20 (d, J=12.3 Hz, 1H), 2.85 (t, J=12.1 Hz, 1H), 2.62 (s, 3H), 2.37 (s, 3H), 2.27 (d, J=11.4 Hz, 1H), 2.21 (s, 3H), 1.97 (t, J=11.4 Hz, 1H), 1.50-1.59 (m, 1H), 1.31-1.38 (m, 1H), 1.27 (t, J=7.1 Hz, 3H), 1.21 (s, 9H), 1.15-1.20 (m, 1H), 1.04 (d, J=12.9 Hz, 1H), 0.90 (s, 3H), 0.58 (s, 3H). LCMS (M+H)=607.4.

### Example 50

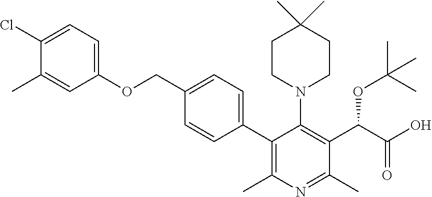 [SMILES: Cc1cc(OCc2ccc(-c3c(C)nc(C)c([C@H](OC(C)(C)C)C(=O)O)c3N3CCC(C)(C)CC3)cc2)ccc1Cl]

### (S)-2-(tert-Butoxy)-2-(5-(4-((4-chloro-3-methylphenoxy)methyl)phenyl)-4-(4,4-dimethylpiperidin-1-yl)-2,6-dimethylpyridin-3-yl)acetic acid

A mixture of (S)-ethyl 2-(tert-butoxy)-2-(5-(4-((4-chloro-3-methylphenoxy)methyl)phenyl)-4-(4,4-dimethylpiperidin-1-yl)-2,6-dimethylpyridin-3-yl)acetate (0.03 g, 0.049 mmol) and LiOH (0.012 g, 0.494 mmol) in 9:1 EtOH/H2O (2 mL) was refluxed for 3 h. Then, cooled and purified by prep-HPLC to afford (S)-2-(tert-butoxy)-2-(5-(4-((4-chloro-3-methylphenoxy)methyl)phenyl)-4-(4,4-dimethylpiperidin-1-yl)-2,6-dimethylpyridin-3-yl)acetic acid.0.33 NH₄OAc (0.026 g, 0.043 mmol, 87% yield) as white solid. ¹H NMR (500 MHz, CDCl₃) δ 7.51 (d, J=8.2 Hz, 2H), 7.28-7.32 (m, 1H), 7.24 (d, J=8.8 Hz, 1H), 7.11-7.15 (m, 1H), 6.90 (d, J=2.7 Hz, 1H), 6.76 (dd, J=3.0, 8.7 Hz, 1H), 5.72 (br. s., 1H), 5.13 (s, 2H), 2.79 (s, 3H), 2.36 (s, 3H), 2.29 (s, 3H), 1.24-1.37 (m, 4H), 1.23 (s, 9H), 0.74 (br. s., 6H). 4H of piperidine were not resolved. LCMS (M+H)=579.4.

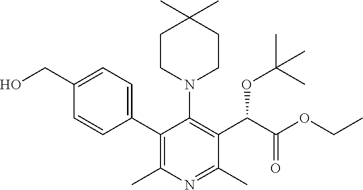 [SMILES: CCOC(=O)[C@@H](OC(C)(C)C)c1c(C)nc(C)c(-c2ccc(CO)cc2)c1N1CCC(C)(C)CC1]

### (S)-Ethyl 2-(tert-butoxy)-2-(4-(4,4-dimethylpiperidin-1-yl)-5-(4-(hydroxymethyl)phenyl)-2,6-dimethylpyridin-3-yl)acetate

Pd(PPh₃)₄ (0.051 g) and K₂CO₃ (0.121 g) were added to a solution of (S)-ethyl 2-(5-bromo-4-(4,4-dimethylpiperidin-1-yl)-2,6-dimethylpyridin-3-yl)-2-(tert-butoxy)acetate (0.200 g) and (4-(hydroxymethyl)phenyl)boronic acid (0.073 g) in dioxane (6 mL) and water (0.7 mL) under nitrogen atmosphere, sealed and heated at 110° C. for 4 h. After cooling, the solvents were removed under vacuum to give a residue which was purified by the preparative HPLC to give (S)-ethyl 2-(tert-butoxy)-2-(4-(4,4-dimethylpiperidin-1-yl)-5-(4-(hydroxymethyl)phenyl)-2,6-dimethylpyridin-3-yl)acetate. LCMS (M+H): 483.4.

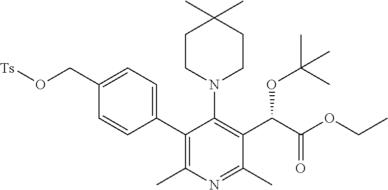 [SMILES: CCOC(=O)[C@@H](OC(C)(C)C)c1c(C)nc(C)c(-c2ccc(COS(=O)(=O)c3ccc(C)cc3)cc2)c1N1CCC(C)(C)CC1]

### (S)-Ethyl 2-(tert-butoxy)-2-(4-(4,4-dimethylpiperidin-1-yl)-2,6-dimethyl-5-(4-((tosyloxy)methyl)phenyl)pyridin-3-yl)acetate

NaH (3.98 mg) was added to a solution of (S)-ethyl 2-(tert-butoxy)-2-(4-(4,4-dimethylpiperidin-1-yl)-5-(4-(hydroxymethyl)phenyl)-2,6-dimethylpyridin-3-yl)acetate (0.040 g) in THF (1.5 mL) at 0° C. The reaction was stirred at room temperature for 1 h, then 4-methylbenzene-1-sulfonyl chloride (0.024 g) was added. The resulting mixture was stirred at room temperature for 18 h. After removal of solvents under vacuum, the crude product was used as is in the following reaction. LCMS (M+H): 637.4.

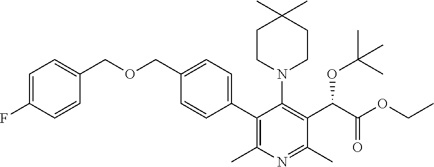 [SMILES: CCOC(=O)[C@@H](OC(C)(C)C)c1c(C)nc(C)c(-c2ccc(COCc3ccc(F)cc3)cc2)c1N1CCC(C)(C)CC1]

### (S)-Ethyl 2-(tert-butoxy)-2-(4-(4,4-dimethylpiperidin-1-yl)-5-(4-(((4-fluorobenzyl)oxy)methyl)phenyl)-2,6-dimethylpyridin-3-yl)acetate

NaH (0.377 mg) was added to a solution of (S)-ethyl 2-(tert-butoxy)-2-(4-(4,4-dimethylpiperidin-1-yl)-2,6-dimethyl-5-(4-((tosyloxy)methyl)phenyl)pyridin-3-yl)acetate (0.010 g) and (4-fluorophenyl)methanol (3.96 mg) in THF (1 mL). The reaction was stirred at room temperature for 1 h. After removal of solvent under vacuum, the crude product was used as is in the following reactions. LCMS(M+H): 591.4.

### Example 51

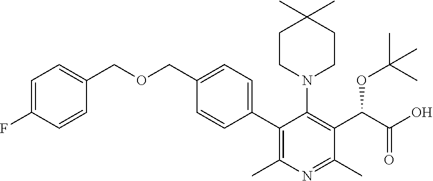 [SMILES: Cc1nc(C)c([C@H](OC(C)(C)C)C(=O)O)c(N2CCC(C)(C)CC2)c1-c1ccc(COCc2ccc(F)cc2)cc1]

### (S)-2-(tert-Butoxy)-2-(4-(4,4-dimethylpiperidin-1-yl)-5-(4-(((4-fluorobenzyl)oxy)methyl)phenyl)-2,6-dimethylpyridin-3-yl)acetic acid

NaOH (2.031 mg) was added to a solution of (S)-ethyl 2-(tert-butoxy)-2-(4-(4,4-dimethylpiperidin-1-yl)-5-(4-(((4-fluorobenzyl)oxy)methyl)phenyl)-2,6-dimethylpyridin-3-yl)acetate (0.010 g) in methanol (0.5 mL) and water (0.5 mL). The reaction was stirred at room temperature for 20 h. Then, solvents were removed under vacuum to give a residue which was purified by the preparative HPLC to give (S)-2-(tert-butoxy)-2-(4-(4,4-dimethylpiperidin-1-yl)-5-(4-(((4-fluorobenzyl)oxy)methyl)phenyl)-2,6-dimethylpyridin-3-yl)acetic acid. LCMS (M+H): 563.4.

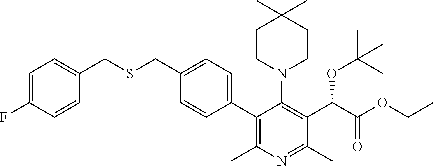 [SMILES: CCOC(=O)[C@@H](OC(C)(C)C)c1c(C)nc(C)c(-c2ccc(CSCc3ccc(F)cc3)cc2)c1N1CCC(C)(C)CC1]

### (S)-Ethyl 2-(tert-butoxy)-2-(4-(4,4-dimethylpiperidin-1-yl)-5-(4-(((4-fluorobenzyl)thio)methyl)phenyl)-2,6-dimethylpyridin-3-yl)acetate

(4-Fluorophenyl)methanethiol (6.70 mg) and NaH (1.507 mg) were added to a solution of (S)-ethyl 2-(tert-butoxy)-2-(4-(4,4-dimethylpiperidin-1-yl)-2,6-dimethyl-5-(4-((tosyloxy)methyl)phenyl)pyridin-3-yl)acetate (0.020 g) in THF (1 mL). The reaction was stirred at room temperature for 1 h, then the reaction was quenched by 1N HCl (5 mL) and ice. The aqueous solution was extracted with EtOAc (4×5 mL). The combined organic layer was dried over MgSO₄. After filtration, the solution was concentrated under vacuum to give a residue which was purified by the preparative HPLC to give (S)-ethyl 2-(tert-butoxy)-2-(4-(4,4-dimethylpiperidin-1-yl)-5-(4-(((4-fluorobenzyl)thio)methyl)phenyl)-2,6-dimethylpyridin-3-yl)acetate. LCMS (M+H): 607.4.

### Example 52

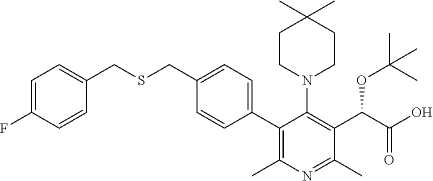 [SMILES: Cc1nc(C)c([C@H](OC(C)(C)C)C(=O)O)c(N2CCC(C)(C)CC2)c1-c1ccc(CSCc2ccc(F)cc2)cc1]

### (S)-2-(tert-Butoxy)-2-(4-(4,4-dimethylpiperidin-1-yl)-5-(4-(((4-fluorobenzyl)thio)methyl)phenyl)-2,6-dimethylpyridin-3-yl)acetic acid

NaOH (4.94 mg) was added to a solution of (S)-ethyl 2-(tert-butoxy)-2-(4-(4,4-dimethylpiperidin-1-yl)-5-(4-(((4-fluorobenzyl)thio)methyl)phenyl)-2,6-dimethylpyridin-3-yl)acetate (0.025 g) in methanol (1 mL) and water (0.5 mL). The reaction was stirred at room temperature for 16 h. Then, solvents were removed under vacuum to give a residue which was purified by the preparative HPLC to give (S)-2-(tert-butoxy)-2-(4-(4,4-dimethylpiperidin-1-yl)-5-(4-(((4-fluorobenzyl)thio)methyl)phenyl)-2,6-dimethylpyridin-3-yl)acetic acid. LCMS (M+H): 579.3.

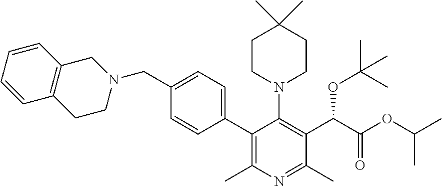 [SMILES: Cc1nc(C)c([C@H](OC(C)(C)C)C(=O)OC(C)C)c(N2CCC(C)(C)CC2)c1-c1ccc(CN2CCc3ccccc3C2)cc1]

### (S)-Isopropyl 2-(tert-butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetate

Pd(PPh₃)₄ (8.37 mg) and Cs₂CO₃ (0.047 g) were added to a solution of (S)-isopropyl 2-(5-bromo-4-(4,4-dimethylpiperidin-1-yl)-2,6-dimethylpyridin-3-yl)-2-(tert-butoxy)acetate (0.034 g) and 2-(4-(4,4,5,5-tetramethyl-1,3,2-dioxaborolan-2-yl)benzyl)-1,2,3,4-tetrahydroisoquinoline (0.030 g) in dioxane (1. mL) and water (0.3 mL), sealed and heated at 105° C. for 3 h. After cooling, the solvents were removed under vacuum to give a residue which was purified by the preparative HPLC to give (S)-isopropyl 2-(tert-butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetate. LCMS (M+H): 612.5.

### Example 53

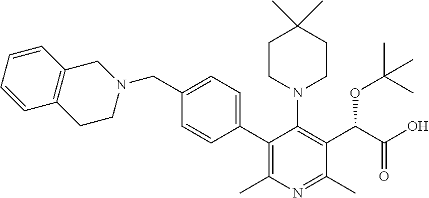 [SMILES: Cc1nc(C)c([C@H](OC(C)(C)C)C(=O)O)c(N2CCC(C)(C)CC2)c1-c1ccc(CN2CCc3ccccc3C2)cc1]

### (S)-2-(tert-Butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetic acid

NaOH (0.018 g) was added to a solution of (S)-isopropyl 2-(tert-butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetate (0.028 g) in methanol (1.2 mL) and water (0.3 mL) sealed and heated at 80° C. for 3 h. After cooling, the solvents were removed under vacuum to give a residue which was purified by the preparative HPLC to give (S)-2-(tert-butoxy)-2-(5-(4-((3,4-dihydroisoquinolin-2(1H)-yl)methyl)phenyl)-4-(4,4-dimethylpiperidin-1-yl)-2,6-dimethylpyridin-3-yl)acetic acid. LCMS (M+H): 570.4.

### Biological Methods

Inhibition of HIV Replication:

A recombinant NL-RLuc proviral clone was constructed in which a section of the nef gene from NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC 18 at the PvuII site. The NL-RLuc virus was prepared by transfection of 293T cells with the plasmid pNLRLuc. Transfections were performed using the LipofectAMINE PLUS kit from Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results from at least 2 experiments were used to calculate the EC₅₀ values. Luciferase was quantitated using the Dual Luciferase kit from Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}](Johnson V A, Byington R T. Infectivity Assay. In Techniques in HIV Research. ed. Aldovini A, Walker B D. 71-76. New York: Stockton Press. 1990). Results are shown in Table 1. Activity equal to A refers to a compound having an EC₅₀≤100 nM, while B and C denote compounds having an EC₅₀ between 100 nM and 1 uM (B) or >1 uM (C).

TABLE 1

| Example | Activity | EC₅₀ μM |
|---|---|---|
| 1 | A | 0.013 |
| 2 | B | 0.113 |
| 3 | A | |
| 4 | A | |
| 5 | A | |
| 6 | B | |
| 7 | A | |
| 8 | A | 0.09 |
| 9 | A | |
| 10 | A | |
| 11 | A | |
| 12 | A | |
| 13 | B | |
| 14 | A | |
| 15 | A | |
| 16 | A | |
| 17 | B | |
| 18 | B | 0.228 |
| 19 | C | 2.073 |
| 20 | A | |
| 21 | A | |
| 22 | A | |
| 23 | A | |
| 24 | A | |
| 25 | A | |
| 26 | A | |
| 27 | A | 0.008 |
| 28 | A | |
| 29 | A | |
| 30 | B | 0.118 |
| 31 | A | |
| 32 | A | |
| 33 | A | |
| 34 | A | |
| 35 | A | |
| 36 | A | |
| 37 | A | |
| 38 | A | |
| 39 | A | |
| 40 | A | |
| 41 | A | 0.015 |
| 42 | A | |
| 43 | A | |
| 44 | A | |
| 45 | A | |
| 46 | A | 0.016 |
| 47 | ND | ND |
| 48 | A | |
| 49 | A | |
| 50 | A | |
| 51 | C | |
| 52 | A | |
| 53 | A | 0.076 |

ND = Not determined

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of Formula I 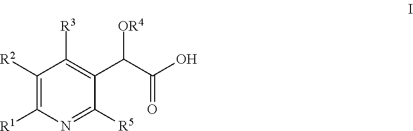
wherein:
R¹ is selected from hydrogen or alkyl;
R² is selected from ((R⁶O)CR⁹R¹⁰)phenyl, ((R⁶S)CR⁹R¹⁰)phenyl, or (((R⁶)(R⁷)N)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is selected from alkyl, cycloalkyl, (cycloalkyl)alkyl, (R⁸)C₁₋₃-alkyl, or (Ar¹)C₀₋₃-alkyl;
R⁷ is selected from hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl;
or N(R⁶)(R⁷) taken together is tetrahydroisoquinolinyl;
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

2. A compound or salt of claim 1 wherein:
R¹ is alkyl;
R² is (((R⁶)(R)N)CR⁹R¹⁰)phenyl;
R³ is piperidinyl substituted with 0-3 substituents selected from cyano, halo, alkyl, haloalkyl, alkoxy, or haloalkoxy;
R⁹ is hydrogen;
R¹⁰ is hydrogen; and
Ar¹ is phenyl substituted with 0-3 substituents selected from halo, alkyl, haloalkyl, alkoxy, haloalkoxy, carboxy, and alkoxycarbonyl.

3. A compound or salt of claim 2 wherein;
R⁶ is (Ar¹)C₁₋₃-alkyl; and
R⁸ is amino, alkylamino, or dialkylamino.

4. A compound of or salt claim 1 wherein R² is ((R⁶O)CR⁹R¹⁰)phenyl or ((R⁶S)CR⁹R¹⁰)phenyl.

5. A compound or salt of claim 1 wherein R² is (((R⁶)(R⁷)N)CR⁹R¹⁰)phenyl.

6. A compound or salt of claim 5 wherein;
R⁶ is (Ar¹)C₀₋₃-alkyl;
R⁷ is hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; and
R⁹ and R¹⁰ are hydrogen.

7. A compound or salt of claim 1 wherein R⁹ and R¹⁰ are hydrogen.

8. A compound of Formula I 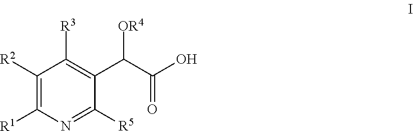
wherein:
R¹ is selected from hydrogen or alkyl;
R² is selected from ((R⁶O)CR⁹R¹⁰)phenyl or ((R⁶S)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is selected from alkyl, cycloalkyl, (cycloalkyl)alkyl, (R⁸)C₁₋₃-alkyl, or (Ar¹)C₀₋₃-alkyl;
R⁷ is selected from hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl;
or N(R⁶)(R⁷) taken together is tetrahydroisoquinolinyl;
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

9. A compound of Formula I 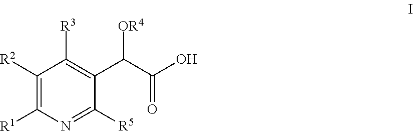
wherein:
R¹ is selected from hydrogen or alkyl;
R² is (((R⁶)(R)N)CR⁹R¹⁰)phenyl;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, alkoxy, or haloalkoxy;
R⁴ is selected from alkyl or haloalkyl;
R⁵ is alkyl;
R⁶ is (Ar)C₀₋₃-alkyl;
R⁷ is hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenylsulfonyl, or mesitylenesulfonyl; and
R⁹ and R¹⁰ are hydrogen.
R⁸ is selected from amino, alkylamino, dialkylamino, azetidinyl, pyrrolidinyl, piperidinyl, piperazinyl, morpholinyl, homopiperidinyl, homopiperazinyl, or homomorpholinyl;
R⁹ is selected from hydrogen or alkyl;
R¹⁰ is selected from hydrogen or alkyl;
or R⁹ and R¹⁰ taken together with the carbon to which they are attached is cycloalkyl;
Ar¹ is a monocyclic heteroaryl or phenyl substituted with 0-3 substituents selected from halo, alkyl, haloalkyl, alkoxy, haloalkoxy, carboxy, and alkoxycarbonyl; and
Ar² is selected from phenyl, furanyl, or thienyl, and is substituted with 0-3 substituents selected from halo, alkyl, haloalkyl, alkoxy, and haloalkoxy;
or a pharmaceutically acceptable salt thereof.

10. A composition useful for treating HIV infection comprising a compound or salt of claim 1 and a pharmaceutically acceptable carrier.

11. The composition of claim 10 further comprising a at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

12. The composition of claim 11 wherein the other agent is dolutegravir.

13. A method for treating HIV infection comprising administering a compound of claim 1, or a pharmaceutically acceptable salt thereof, to a patient in need thereof.

14. The method of claim 13 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

15. The method of claim 14 wherein the other agent is dolutegravir.

16. The method of claim 15 wherein the other agent is administered to the patient prior to, simultaneously with, or subsequently to the compound of claim 1.
